(19) United States
(12) Patent Application Publication
(10) Pub. No.: US 2022/0074956 A1
(43) Pub. Date: Mar. 10, 2022

(54) METHODS FOR DETERMINING THE HEMATOCRIT LEVEL IN A SAMPLE OF WHOLE BLOOD

(71) Applicant: Gentian AS, Moss (NO)

(72) Inventors: Camilla Fant, Kullavik (SE); Kathrin Sunde, Moss (NO); Erling Sundrehagen, Moss (NO); Olov Wahlsten, Göteborg (SE)

(21) Appl. No.: 17/416,151

(22) PCT Filed: Dec. 19, 2019
(86) PCT No.: PCT/EP2019/086468

§ 371 Date: Jun. 18, 2021

(30) Foreign Application Priority Data
Dec. 19, 2018 (SE) 1851609-6
May 13, 2019 (SE) 1950567-6

(51) Int. Cl. G01N 33/72; G01N 33/558; G01N 21/3577

(52) U.S. Cl. CPC G01N 33/721; G01N 21/3577; G01N 33/558

(57) ABSTRACT

A hematocrit level in a sample of whole blood can be determined in a lateral flow assay setting, where a sample of whole blood is applied to a substrate on which a blood spot is formed; taking an image of said blood spot within 1-300 seconds from application, subjecting said image to image analysis, and determining the hematocrit level based on a value of at least one parameter extracted from said image. Such reagent-free hematocrit measurement can be integrated in lateral flow assay devices for the measurement of an analyte and contribute to significantly improved precision of such assays.

### TECHNICAL FIELD

This disclosure relates to the field of clinical analysis of whole blood samples, and to methods for determining the amount of hemoglobin and/or the hematocrit level, i.e. the volume fraction of red blood cells in a sample of whole blood, and in particular to a rapid method for determination of the hemoglobin and/or the hematocrit level of samples of whole blood with simple, affordable equipment and without the use of any reagents. The disclosure also relates to lateral flow assay methods and devices for analysing a sample of whole blood and quantitatively determining the concentration of an analyte in plasma, with consideration of the hematocrit level of the whole blood sample.

### BACKGROUND

Blood tests are a cornerstone of modern medicine, and it is today possible to determine the presence and concentration of literally hundreds of analytes. Blood tests are used to determine the physiological and biochemical status of a patient, and the results are central in determining nutritional status, health, the presence or absence of various diseases, the effectiveness of a treatment, organ function and also for example to detect drug abuse. Methods and devices for this purpose range from comparatively simple test strips to analysis robots, capable of holding reagents for performing hundreds of tests on a single sample.

Whole blood is comprised of erythrocytes, platelets and leukocytes suspended in plasma. In blood from healthy individuals, the erythrocytes constitute the clear majority of cells; the erythrocytes containing hemoglobin (Hb), which gives blood its red colour and which has oxygen-binding abilities. Plasma mainly consists of water (approx. 93%) but also of salts, various proteins and lipids as well as other constituents, e.g. glucose. The plasma also contains trace amounts of hundreds if not thousands of biochemical compounds out of which a great number are established clinical analytes, while others are still the subject of investigation. Tests or assays conducted in a laboratory setting are often based on plasma or serum. For Point-Of-Care (POC) applications, it is however preferable if a sample of whole blood can be used, avoiding the step of separating the serum or plasma prior to applying the sample to the assay.

### Hemoglobin

Hemoglobin (Hb) is an iron-containing metalloprotein responsible for the transport of oxygen in the red blood cells of all vertebrates. In mammals, Hb makes up about 96 weight-% of the red blood cells' dry content and about 35 weight-% of the total content.

Methods for the measurement of Hb were first developed more than a century ago, making this one of the first diagnostic blood tests available to clinicians. Today, Hb concentration measurement is among the most commonly performed blood tests, usually as part of a complete blood count. For example, it is typically tested before or after blood donation. Results are reported in g/L, sometimes in g/dL or mol/L. Normal levels are 14 to 18 g/dl for men, 12 to 16 g/dl for women (11-14 g/dl for pregnant women), and 11-16 g/dl for children (Henny H. Billett, Chapter 151, Hemoglobin and Hematocrit, in Clinical Methods: The History, Physical and Laboratory Examinations, Walker H K, Hall W D, Hurst J W, eds., Boston: Butterworths; 1990. 3rd edition).

If the concentration is below normal, this is called anaemia. Anaemia can be due to blood loss, decreased red blood cell production, and increased red blood cell breakdown. In addition to blood donations, causes of blood loss include trauma and gastrointestinal bleeding, among others. Decreased production of red blood cells can be caused by iron deficiency, a lack of vitamin B12, thalassemia, and a number of neoplasms of the bone marrow. Increased breakdown of red blood cells can be due to a number of genetic conditions such as sickle cell anaemia, infections like malaria, and certain autoimmune diseases.

The hemiglobincyanide (HiCN) test originally developed in the 1950-ties remains the recommended method of the International Committee for Standardization in Haematology (ICSH) against which all new methods for the measurement of the concentration of Hb are judged and standardized. ICSH was founded in 1966 and has been active ever since, establishing and revising recommendations for the standardised HiCN method and organizing and supervising production and distribution of an international reference preparation, a sterile ampouled HiCN solution of exactly known concentration.

Portable hemoglobinometers such as the HemoCue® 201 device (HemoCue AB, Sweden) allow accurate determination of hemoglobin in a Point-of-Care setting, and at blood donation centres. The devices are essentially photometers which allow measurement of the color intensity of solutions.

The measurements are made in disposable microcuvettes, which also act as reaction vessels. The reagents necessary for both the release of Hb from erythrocytes and for the conversion of Hb to a stable coloured product are present in dried form on the walls of the cuvette. All that is required is introduction of a small sample (typically 10 μL) of capillary, venous or arterial blood to the microcuvette and insertion of the microcuvette into the instrument. The instrument is factory pre-calibrated using the above mentioned HiCN standard, and the absorbance of the test solution is automatically converted to the concentration of total hemoglobin (ctHb). The result is displayed in less than a minute.

Reagent-less analyzers have been on the market since 2006 (the HemoCue® 301) and recently, the DiaSpect Tm (EKF Diagnostics plc/DiaSpect Medical GmbH) was introduced, using a reagentless cuvette, a photometric method, and providing results in approximately one second.

### Hematocrit

The volume fraction of packed red blood cells in a blood sample is referred to as the hematocrit (Hct) and expressed as % of the total sample volume. Normal Hct levels are rather constant, in the range of 40% to 54% for adult males and 36% to 48% for adult women (Henny H. Billett, 1990, ibid). Deviations from these reference levels are generally regarded as the sign of a critical disease such as anaemia, leukaemia, a kidney infection, or a diet deficiency; but may also be an indication of an unambiguous condition, such as pregnancy, or even extensive exercise.

The interference of Hct is also considered to be an important issue when measuring the concentration of different analytes in whole blood, plasma or serum. If the concentration of an analyte which is present only in plasma is given in relation to the volume or weight of a sample of whole blood, the Hct needs to be considered. Variations in Hct can otherwise cause serious errors in all qualitative and quantitative clinical blood analysis assays.

To illustrate the significance of knowing the Hct, one can consider two samples of whole blood, one obtained from a patient having low Hb, 100 g/l and a sample from a patient having very high Hb, 180 g/l, both values being physiologically relevant. The first patient will have a Hct of approximately 30% while the second patient has a Hct of approx. 56%. For a sample size of 25 μl, this means that for the first patient, there will be 17.5 μl plasma available for analysis, and for the second patient, only 11 μl. When determining the concentration of an analyte present in plasma, these variations have considerable significance.

It is well known that quantitative colorimetric determination of Hb can be performed at 540 nm after first oxidizing hemoglobin and its derivatives (except sulfhemoglobin) to methemoglobin in the presence of an alkaline potassium ferricyanide and potassium cyanide solution (Drabkin's reagent). Methemoglobin reacts with potassium cyanide to form cyanomethemoglobin, which has a maximum absorption at 540 nm. The colour intensity measured at 540 nm is proportional to the total hemoglobin concentration.

A paper-based test for measuring Hb has also been disclosed in 2013 by Yang et al. (Yang et al., Simple Paper-Based test for Measuring Blood Hemoglobin Concentration in Resource-Limited Settings, Clinical Chemistry, (2013) 59:10, 1506-1513). In this test, a 20 μL droplet of a mixture of blood and Drabkin's reagent is deposited onto patterned chromatography paper. The resulting blood stain is left to dry for 25 minutes, scanned and the digital images analysed based on a red/green/blue (RGB) colour model. The green channel showed the best linear fit and was selected to quantify Hb in the blood samples.

A method for Hct prediction using non-contact diffuse reflectance spectroscopy has been presented (Capiau, et al., A Novel, Non-destructive, Dried Blood Spot-Based Hematocrit Prediction Method Using Noncontact Diffuse Reflectance Spectroscopy, Analytical Chemistry. 2016, 88, 6538-6546). The results indicated that mere scanning of a dried blood spot (DBS) suffices to derive its approximate Hct. Venous blood was collected from consenting healthy volunteers in blood collection tubes with lithium heparin as anticoagulant. Dried blood spots (DBSs) were prepared at the day of blood collection by depositing 25 μL of blood onto Whatman 903 filter paper. The blood spots were always allowed to dry at ambient conditions for at least 2 h. The obtained DBSs were either analysed immediately after drying or stored in zip-locked plastic bags in the presence of a desiccant until analysis. The DBSs were found to be stable for at least 5 months at room temperature, and at least up to 3 days at elevated temperatures (60° C.). The DBSs were illuminated using a 10 W tungsten-halogen light source, and a spectrometer recorded the wavelength dependence of the reflected light intensity between 354 and 1042 nm.

An analysis technique using a histogram for the colorimetric quantification of blood hematocrit, was proposed in 2017, and the researchers developed a smartphone-based “histogram app” for the detection of hematocrit integrating the smartphone embedded camera with a microfluidic chip via a custom-made optical platform (Jalal et al., Histogram analysis for smartphone-based rapid hematocrit determination, Biomed Opt Express. 2017 Jul. 1; 8(7): 3317-3328).

U.S. Pat. No. 8,730,460 (Yan et al., Paper Based Spectrophotometric Detection of Blood Hemoglobin Concentration) discloses a paper based spectrophotometric detection of blood Hb concentration, wherein spectrophotometric techniques are used to measure light transmission at specified wavelengths through a paper medium containing a blood sample. The light transmission information is then used in the calculation of blood Hb concentration. In certain embodiments, the paper medium may be chemically treated to lyse the blood sample prior to measurement of the light transmission information.

WO 2017/087834 presents a general concept of a multiplex diagnostic assay cartridge for detection of a plurality of target molecules. One embodiment relates to a multiplex diagnostic assay cartridge having a pre-processing module and—distal to the sample addition well—parallel assay regions for a ferritin immunoassay, a C-reactive protein immunoassay region and a hemoglobin colorimetric assay region.

### SUMMARY

The present inventors have surprisingly found that the hematocrit level and/or hemoglobin concentration in a sample of whole blood can be determined rapidly and yet accurately by measuring the reflectance of said sample, when applied to a substrate. The measurement can be performed very soon and even substantially immediately after application of the sample to the substrate, without waiting for the blood sample to dry. The present inventors have also found that the measurement of Hct in or in parallel to a lateral flow assay for the determination of the concentration of an analyte in plasma and taking the measured Hct into account when calculating the concentration of the analyte, significantly improves the accuracy of the result.

Accordingly, a first aspect of the present description concerns an optical method for determining a hematocrit level in a sample of whole blood in a lateral flow assay device, wherein the method comprises the steps of (i) applying the sample to a substrate to form a blood; (ii) taking an image of said blood spot within 1-300 seconds after the applying step; (iii) analysing said to extract at least one parameter; and (iv) determining the hematocrit level based on a value of the at least one extracted parameter.

According to an embodiment of the above aspect, the sample of whole blood is an untreated sample.

According to another embodiment of the above aspect, freely combinable with other aspects and embodiments, the image is taken within 1-180 seconds, preferably 1-120 seconds, more preferably within 1-30 seconds, and most preferably within 1-10 seconds after the applying step.

According to yet another embodiment of the above aspect, freely combinable with other aspects and embodiments, said at least one extracted parameter is a reflectance of said blood spot or an area of said blood spot. Preferably both the reflectance of said blood spot and the area of said blood spot are determined and then correlated to a preliminary hematocrit level, and the average of the two is used as a measure (value) of the hematocrit level.

According to yet another embodiment of the above aspect, freely combinable with other aspects and embodiments, the reflectance value is determined at at least one wavelength in a range from 390 nm to 1000 nm, preferably in the interval of 650 nm to 1000 nm, for example at at least one wavelength chosen from 660 nm, 780 nm, 800 nm, and 940 nm. Preferably the reflectance is determined at 800 nm, or determined at both 660 nm and 940 nm.

According to an embodiment, freely combinable with other aspects and embodiments, reflectance is measured as the median intensity of the pixels included in said image taken in step (ii) using an 800 nm optical filter.

According to another embodiment, also freely combinable with other aspects and embodiments, the method further comprises a calibration step by means of which a reference hematocrit level of a reference sample is determined by centrifugation.

According to yet another embodiment, also freely combinable with other aspects and embodiments, a hematocrit level is determined by first optically determining a concentration of hemoglobin in said sample and then converting said hemoglobin concentration into thee hematocrit level. Preferably the hemoglobin concentration is converted into the hematocrit level by multiplying the hemoglobin concentration in g/dl by a factor of 3, thus yielding the hematocrit level in %.

A second aspect of the present disclosure relates to a lateral flow assay method for determining the concentration of an analyte in a sample of whole blood, comprising the following steps:
a) applying an untreated sample of whole blood to a first surface in said lateral flow assay to form a blood spot thereon;
b) taking an image of said blood spot within 1-300 seconds after the applying step;
c) analysing said image to determine a first value indicative of the hematocrit level of said sample;
d) determining a second value indicative of the amount of the analyte in the sample; and
e) determining the concentration of the analyte in the sample based on the hematocrit level determined in step c) and the amount of analyte determined in step d).

According to an embodiment of the second aspect, the analyte is chosen from ferritin, transferrin, plasma calprotectin, C-reactive protein (CRP), cystatin C, plasma procalcitonin (PCT) and anti-CCP antibodies.

According to yet another embodiment of the second aspect, also freely combinable with all other embodiments of said aspect, said image is taken within 1-180 seconds, preferably 1-120 seconds, more preferably within 1-30 seconds, and most preferably within 1-10 seconds after the applying step (a).

According to another embodiment of the second aspect, freely combinable with all other embodiments of said aspect, said at least one parameter is the reflectance of said blood spot or an area of said blood spot. Preferably the reflectance of said blood spot and the area of said blood spot are determined and then correlated to a preliminary hematocrit level, and the average of the two is used as a measure (value) of the hematocrit level.

According to another embodiment of the second aspect, freely combinable with all other embodiments of said aspect, the reflectance value is determined at at least one wavelength in a range from 390 nm to 1000 nm, preferably in the interval of 650 nm to 1000 nm, for example at at least one wavelength chosen from 660 nm, 780 nm, 800 nm, and 940 nm. Preferably the reflectance is determined at 800 nm, or determined at both 660 nm and 940 nm.

According to an embodiment of the second aspect, freely combinable with other aspects and embodiments reflectance is measured as the median intensity of the pixels included in an image taken in step (b) using an 800 nm optical filter.

According to another embodiment, also freely combinable with other aspects and embodiments, the method comprises a calibration step by means of which a reference hematocrit level of a reference sample is determined by centrifugation.

According to yet another embodiment, also freely combinable with other aspects and embodiments, a hematocrit level is determined by first optically determining a concentration of hemoglobin in said sample and then converting said hemoglobin concentration to a hematocrit level. Preferably the hemoglobin concentration is converted into the hematocrit level by multiplying the hemoglobin concentration in g/dl by a factor of 3, thus yielding the hematocrit level in %.

A third aspect of the present disclosure relates to a system for determining the hematocrit in a sample of whole blood, wherein said system comprises a lateral flow assay device having a substrate configured to form a blood spot thereon upon application of a sample of whole blood onto said substrate, at least one light source, a detector arranged to detect light reflected from said blood spot and to determine the reflectance and/or size of said blood spot, and a processor configured to correlate the reflectance and/or the size of the blood spot to a hematocrit level of said sample based on stored values of reflectance and/or size obtained from known hematocrit levels.

A fourth aspect relates to a system for determining the hematocrit in a sample of whole blood, wherein said system comprises a lateral flow assay having a substrate configured to form a blood spot thereon upon application of a sample of whole blood onto said substrate, at least one light source, a detector arranged to detect light reflected from said blood spot and to determine the reflectance and/or size of said blood spot, and a processor configured to correlate the reflectance and/or the size of the blood spot to a hemoglobin concentration of said sample based on stored values of reflectance and/or size obtained from known hemoglobin concentrations, and to calculate the hematocrit level based on said hemoglobin concentration.

One aspect relates to a lateral flow assay device for determining the concentration of plasma calprotectin in a whole blood sample, wherein said device comprises a substrate configured to form a blood spot thereon upon application of a sample of whole blood onto the substrate, a conjugate pad with anti-calprotectin antibodies conjugated to a marker, a membrane with at least one test line of immobilized anti-calprotectin antibodies, and an absorbent pad.

Another aspect relates to a lateral flow assay device for determining the concentration of cystatin C in a whole blood sample, wherein said device comprises a substrate configured to form a blood spot thereon upon application of a sample of whole blood onto the substrate, a conjugate pad with anti-cystatin C antibodies conjugated to a marker, a membrane with at least one test line of immobilized anti-cystatin C antibodies, and an absorbent pad.

Yet another aspect relates to a lateral flow assay device for determining the concentration of ferritin in a whole blood sample, wherein said device comprises a substrate configured to form a blood spot thereon upon application of a sample of whole blood onto the substrate, a conjugate pad with anti-ferritin antibodies conjugated to a marker, a membrane with at least one test line of immobilized anti-ferritin antibodies, and an absorbent pad.

Another aspect relates to a lateral flow assay device for determining the concentration of plasma procalcitonin in a whole blood sample, wherein said device comprises a substrate configured to form a blood spot thereon upon application of a sample of whole blood onto the substrate, a conjugate pad with anti-procalcitonin antibodies conjugated to a marker, a membrane with at least one test line of immobilized anti-procalcitonin antibodies, and an absorbent pad.

Another aspect relates to a lateral flow assay device for determining the concentration of C-reactive protein (CRP) in a whole blood sample, wherein said device comprises a substrate configured to form a blood spot thereon upon application of a sample of whole blood onto the substrate, a conjugate pad with anti-CRP antibodies conjugated to a marker, a membrane with at least one test line of immobilized anti-CRP antibodies, and an absorbent pad.

Another aspect relates to a lateral flow assay device for determining the concentration of anti-CCP antibodies in a whole blood sample, wherein said device comprises a substrate configured to form a blood spot thereon upon application of a sample of whole blood onto the substrate, a conjugate pad with one or more cyclic citrullinated peptides (CCP) conjugated to a marker, a membrane with at least one test line of immobilized antibodies, and an absorbent pad.

In an embodiment of a lateral flow assay device according to any one of the aspects above, said substrate is arranged in fluid connection with the conjugate pad of said lateral flow assay, and wherein said substrate is a glass fiber-based filter.

In another embodiment of a lateral flow assay device according to any one of the aspects above, said substrate is arranged parallel to and not in fluid connection with the conjugate pad of said lateral flow assay, and wherein said substrate is chosen from a glass fiber-based filter, a cellulose-based filter, and a substrate having an impermeable surface.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

The invention is now described, by way of example, with reference to the accompanying drawings, in which:

FIG. 1 schematically shows an experimental set-up of the equipment including a camera, sample holder, lamps etc, representative for the different set-ups used in the examples.

FIG. 2 illustrates how the relevant pixels are identified starting from a photographic image of a blood spot. Based on the contrast between the blood spot and the background, the spot is masked, and the relevant pixels identified for further analysis.

FIG. 3 shows a schematic cross-section of a lateral flow device according to an embodiment of the invention, comprising a portion for receiving a sample of whole blood, such as a sample pad (1) or a substrate, arranged in fluid connection with further substrates, media or filters (2, 3, 6) arranged on a support or backing (10).

FIG. 4 shows a schematic cross-section of a device according to an embodiment of the invention, where a membrane (100) is arranged on the sample pad (1). Such membrane can have the function of separating red blood cells from plasma with no or minimal hemolysis.

FIG. 5 shows a schematic cross-section of a device according to another embodiment of the invention, wherein a sample is added to receiving means (12), for example a recess in a support or backing (11), from which receiving means said sample comes into fluid communication with further media or filters arranged on said backing (11).

FIG. 6 shows a schematic view from above of a device according to the embodiment illustrated in FIG. 3.

FIG. 7 shows a schematic view from above of a device according to the embodiment illustrated in FIG. 4, wherein a membrane (100) is arranged on the filter or sample pad (1).

FIG. 8 shows a schematic view from above of a device according to the embodiment illustrated in FIG. 5.

FIG. 9 shows a schematic view from above of a device according to another embodiment comprising separate receiving means (13) arranged in parallel to the flow path of the lateral flow assay device, and a first medium or sample pad (1), where said medium or filter is in fluid connection with further mediums or filters on a support (20). Said separate receiving means can be a recess, a delimited area on the support or backing (10, 11, 20), or a filter paper, such as a cellulose-based filter paper or a glass fibre-based filter paper.

FIG. 10 is a Bland Altman plot showing the results of optical hemoglobin measurements on one type of filter paper. The difference (g/dl) and the mean of the measurements (g/dl) are indicated on the y- and x-axis, respectively.

FIG. 11 is another Bland Altman plot showing the results of optical hemoglobin measurements on another type of filter paper. The difference (g/dl) and the mean of the measurements (g/dl) are indicated on the y- and x-axis, respectively.

FIG. 12 illustrates an intermediate product used in the assembly of a lateral flow test, having a support or backing (10), and media or filters (1), (2), (3) and (6) arranged on said support. When this intermediate product is cut crosswise, lateral flow assay strips are formed, where (1) corresponds to the sample addition pad, (2) is the conjugate pad, (3) is the filter with the test line and control line, and (6) is the absorbent pad or wicking pad, all arranged on a support or backing (10).

FIG. 13 is an exploded view of a prototype assay device, showing a housing (200), enclosing inter alia the filters or media (1), (2), (3) and (6), and having a sample port (201) and one or more openings (202) exposing the test line and control line, (4) and (5) respectively.

FIG. 14 A through D show the correlation of hemoglobin concentration versus signal (the median pixel intensity of the blood spot after background correction) for different wavelengths; 543 nm (A), 590 nm (B), 660 nm (C), and 940 nm (D).

FIG. 15 is a graph showing how the mode-of-fit parameter extracted from the pixel intensities correlates with Hb concentration (g/I) for six different samples tested on four different filter mediums using a 660 nm optical filter.

FIG. 16 is a graph showing how the mode-of-fit parameter extracted from the pixel intensities correlates with hematocrit volume fraction (%) for six different samples tested on four different filter mediums using a 660 nm optical filter

FIG. 17 is a graph showing how the mode-of-fit parameter extracted from the pixel intensities correlates with Hb concentration (g/l) for 18 different samples tested on four different filter mediums using a 660 nm optical filter. The curves are in the following order, from top to bottom: the Whatman™ 17 Chr filter paper, the Whatman® 2668 cellulose chromatography paper, a glass fiber-based filter GF/DVA and a glass fiber-based filter VF2, both from GE Healthcare.

FIG. 18 is a graph showing how the mode-of-fit parameter extracted from the pixel intensities correlates with the hematocrit volume fraction (%) for 18 different samples tested on four different filter mediums (same as in FIGS. 15-17) using a 660 nm optical filter. The curves for the different filter papers appear also here in the same order.

FIG. 19 is a graph showing the Pearson correlation (Mode-of-fit versus Hb) for the four different filter mediums tested at six different wavelengths; 543.5 nm, 590 nm, 660 nm, 780 nm, 800 nm, and 940 nm.

FIG. 20 is a graph showing the Pearson correlation (Median versus Hb) as a function of time (15-300 sec) for six different wavelengths; 543.5 nm, 590 nm, 660 nm, 780 nm, 800 nm, and 940 nm.

FIG. 21 is a graph showing the median pixel value as a function of Hb concentration, measured at 15 seconds after addition of a whole blood sample, and using a 780 nm optical filter. A second-degree polynomial fit was used to formulate a prediction model.

FIG. 22 is a graph showing the Hb predictability over time, measured as average difference between true and assigned Hb concentration (g/I) as a function of time after blood addition (15-300 sec), using the prediction model calculated from the previous graph.

FIG. 23 shows the correlation between the calprotectin concentration determined with a modified lateral flow test, using whole blood, and a turbidimetric assay, using plasma. For the whole blood test either a constant Hct level of 44.5% (filled circles) or a Hct level predicted optically (open circles) was applied.

FIG. 24 shows the relative deviation for calprotectin in whole blood determined using a modified lateral flow test and adjusted for an assumed Hct of 44.5%, compared to turbidimetric measurement in plasma.

FIG. 25 shows the relative deviation for calprotectin in whole blood determined using a modified lateral flow test and adjusted for the Hct, predicted optically, compared to turbidimetric measurement in plasma, the results confirming that a whole blood lateral flow test for calprotectin gives accurate results when the Hct level is predicted and accounted for.

FIG. 26 shows the correlation between Hct and median pixel value at 800 nm fitted to a 3^{rd} degree polynomial.

FIG. 27 shows the correlation between Hct and blood spot area fitted to a 3^{rd} degree polynomial.

FIG. 28 shows the improved accuracy achieved when combining the determination of Hct based on median pixel intensity and area of blood spot.

### DETAILED DESCRIPTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

The term “sample” as in “a sample of whole blood” refers to a sample taken from a human or animal body, and which sample will not be returned to said human or animal body.

The term “whole blood”, as used herein, refers to blood with all its constituents. In other words, whole blood comprises both blood cells such as erythrocytes, leukocytes, and thrombocytes, and blood plasma in which the blood cells are suspended.

The term “blood plasma” (or “plasma”), as used herein, denotes the blood's liquid medium and is an substantially aqueous solution containing water, blood plasma proteins, and trace amounts of other materials such as serum albumin, blood clotting factors, immunoglobulins (antibodies), hormones, carbon dioxide, various other proteins and various electrolytes (mainly sodium and chloride).

The term “blood serum” (or “serum”), as used herein, refers to plasma from which the clotting proteins have been removed.

In further embodiments, the sample applied onto the substrate is an untreated whole blood sample. The term “untreated”, as used herein, is to be understood that after collecting the sample (e.g., by blood withdrawal from a patient) and before subjecting it to the inventive methods, no further sample processing (e.g., fractionation methods, drying the whole blood, e.g. on filter paper, for sample storage, and reconstitution of dried blood samples by re-dissolving in water, and the like) occurs.

However, the storage of the samples per se, for example in a refrigerator or freezer, is not to be considered a processing step as defined above. Thus, the sample may be applied onto the substrate immediately after collection or it may be introduced into the device after storage of the sample for one or more hours to one or more days or weeks.

In addition, since whole blood samples comprise blood-clotting factors, which will cause the formation of blood clots upon prolonged storage of the samples and whose presence may thus interfere with the subsequent analysis, the addition of anti-coagulants (i.e. inhibitors of blood clotting) is also not a treatment of the sample within the meaning of the present invention. Multiple compounds acting as anti-coagulants are well known in the art. Examples of anti-coagulants include inter alia natural or synthetic (i.e. obtained by chemical synthesis and/or recombinant DNA technology) vitamin K antagonists, natural or synthetic direct thrombin inhibitors, citrate, oxalate, heparin and ethylene-diamine-tetra acetic acid (EDTA).

In other embodiments, the whole blood sample is applied onto the substrate directly (i.e. in untreated form, as defined above) from a subject. Particularly, the whole blood sample may be obtained from a puncture at a fingertip of the subject. For example, after puncturing the fingertip, the leaking blood may be collected by contacting the blood with a capillary such that the blood is introduced by capillary force without external manipulation. The capillary may then be positioned relative to the assay device employed such that the blood can pass or can be actively transferred into the device. Alternatively, the punctured fingertip may be positioned immediately adjacent to one of the openings of the device, which are detailed below (e.g. by pressing the fingertip directly on such an opening) such that the blood leaking from the puncture may be introduced into the device.

There are standardized methods for obtaining and handling a blood sample taken from a human or animal body, involving the use of needles, syringes, micro cuvettes etc. These methods are well-known to persons skilled in the art. The currently most preferred type of sample is lithium heparin treated sample of whole blood. There are several blood collection tubes containing spray-coated lithium heparin readily available from various commercial supplies, e.g. the BD Vacutainer® available from BD, Oakville, Ontario, Canada.

The term “substrate” refers to any substrate capable of receiving a sample of whole blood for analysis, preferably a flat substrate of homogenous colour, and most preferably a fibrous substrate, such as a cellulose-based or glass fiber-based filter.

The term “hematocrit value” or “hematocrit level” refers to the volume percentage (vol %) of red blood cells (RBC) in blood. The measurement depends on the number and size of red bloods cells, and varies with gender, age, and medical condition. It is normally about 40-54 for adult men and 36% to 48% for adult women. Because the purpose of red blood cells is to transfer oxygen from the lungs to body tissues, a blood sample's hematocrit level—the red blood cell volume percentage—can become a point of reference of its capability of delivering oxygen. Hematocrit levels that are too high or too low can indicate a blood disorder, dehydration, or other medical conditions. An abnormally low hematocrit level may suggest anaemia, a decrease in the total amount of red blood cells, while an abnormally high hematocrit is called polycythemia.

The term “analyte” in this disclosure refers to any and all clinically relevant analytes present in blood and plasma, for example antibodies, hormones and proteins, for example but not limited to ferritin, plasma calprotectin, cystatin C, procalcitonin, and C-reactive protein. Examples of antibodies include autoantibodies as well as antibodies against infectious agents such as virus and bacteria, for example anti-CCP, anti-streptolysin-O, anti-HIV, anti-hepatitis (anti-HBc, anti-HBs etc), antibodies against Borrelia, and specific antibodies against microbial proteins.

A first aspect of the present description concerns an optical method for determining a hematocrit level in a sample of whole blood in a lateral flow assay device, wherein the method comprises the steps of (i) applying the sample to a substrate to form a blood; (ii) taking an image of said blood spot within 1-300 seconds after the applying step; (iii) analysing said to extract at least one parameter; and (iv) determining the hematocrit level based on a value of the at least one extracted parameter.

According to an embodiment of the above aspect, the sample of whole blood is an untreated sample. Untreated here means that no reagents have been added to the sample. Preferably there are also no reagents added to, or immobilized in the substrate or on the surface onto which the sample is added.

According to another embodiment of the above aspect, freely combinable with other aspects and embodiments, the image is taken within 1-180 seconds, preferably 1-120 seconds, more preferably within 1-30 seconds, and most preferably within 1-10 seconds after the applying step. In a Point-of-Care application, it desirable that a result can be obtained quickly. It is therefore a significant advantage that, in the present method, a reading can be performed already within seconds from the application of the sample to the lateral flow device.

According to yet another embodiment of the above aspect, freely combinable with other aspects and embodiments, said at least one extracted parameter is a reflectance of said blood spot or an area of said blood spot. Preferably both the reflectance of said blood spot and the area of said blood spot are determined and then correlated to a preliminary hematocrit level, and the average of the two is used as a measure (value) of the hematocrit level.

According to yet another embodiment of the above aspect, freely combinable with other aspects and embodiments, the reflectance value is determined at at least one wavelength in a range from 390 nm to 1000 nm, preferably in the interval of 650 nm to 1000 nm, for example at at least one wavelength chosen from 660 nm, 780 nm, 800 nm, and 940 nm. Preferably the reflectance is determined at 800 nm, or determined at both 660 nm and 940 nm.

According to an embodiment, freely combinable with other aspects and embodiments, reflectance is measured as the median intensity of the pixels included in said image taken in step (ii) using an 800 nm optical filter.

According to another embodiment, also freely combinable with other aspects and embodiments, the method further comprises a calibration step by means of which a reference hematocrit level of a reference sample is determined by centrifugation.

According to yet another embodiment, also freely combinable with other aspects and embodiments, a hematocrit level is determined by first optically determining a concentration of hemoglobin in said sample and then converting said hemoglobin concentration into thee hematocrit level. Preferably the hemoglobin concentration is converted into the hematocrit level by multiplying the hemoglobin concentration in g/dl by a factor of 3, thus yielding the hematocrit level in %.

A second aspect of the present disclosure relates to a lateral flow assay method for determining the concentration of an analyte in a sample of whole blood, comprising the following steps:
(a) applying an untreated sample of whole blood to a first surface in said lateral flow assay to form a blood spot thereon;
(b) taking an image of said blood spot within 1-300 seconds after the applying step;
(c) analysing said image to determine a first value indicative of the hematocrit level of said sample;
(d) determining a second value indicative of the amount of the analyte in the sample; and
(e) determining the concentration of the analyte in the sample based on the hematocrit level determined in step c) and the amount of analyte determined in step d).

According to an embodiment of the second aspect, the analyte is chosen from ferritin, transferrin, plasma calprotectin, C-reactive protein (CRP), cystatin C, plasma procalcitonin (PCT) and anti-CCP antibodies.

According to yet another embodiment of the second aspect, also freely combinable with all other embodiments of said aspect, said image is taken within 1-180 seconds, preferably 1-120 seconds, more preferably within 1-30 seconds, and most preferably within 1-10 seconds after the applying step (a).

According to another embodiment of the second aspect, freely combinable with all other embodiments of said aspect, said at least one parameter is the reflectance of said blood spot or an area of said blood spot. Preferably the reflectance of said blood spot and the area of said blood spot are determined and then correlated to a preliminary hematocrit level, and the average of the two is used as a measure (value) of the hematocrit level.

According to another embodiment of the second aspect, freely combinable with all other embodiments of said aspect, the reflectance value is determined at at least one wavelength in a range from 390 nm to 1000 nm, preferably in the interval of 650 nm to 1000 nm, for example at at least one wavelength chosen from 660 nm, 780 nm, 800 nm, and 940 nm. Preferably the reflectance is determined at 800 nm, or determined at both 660 nm and 940 nm.

According to an embodiment of the second aspect, freely combinable with other aspects and embodiments reflectance is measured as the median intensity of the pixels included in an image taken using an 800 nm optical filter.

According to another embodiment, also freely combinable with other aspects and embodiments, the method comprises a calibration step by means of which a reference hematocrit level of a reference sample is determined by centrifugation.

According to yet another embodiment, also freely combinable with other aspects and embodiments, a hematocrit level is determined by first optically determining a concentration of hemoglobin in said sample and then converting said hemoglobin concentration to a hematocrit level. Preferably the hemoglobin concentration is converted into the hematocrit level by multiplying the hemoglobin concentration in g/dl by a factor of 3, thus yielding the hematocrit level in %.

A third aspect of the present disclosure relates to a system for determining the hematocrit in a sample of whole blood, wherein said system comprises a lateral flow assay device having a substrate configured to form a blood spot thereon upon application of a sample of whole blood onto said substrate, at least one light source, a detector arranged to detect light reflected from said blood spot and to determine the reflectance and/or size of said blood spot, and a processor configured to correlate the reflectance and/or the size of the blood spot to a hematocrit level of said sample based on stored values of reflectance and/or size obtained from known hematocrit levels.

A fourth aspect relates to a system for determining the hematocrit in a sample of whole blood, wherein said system comprises a lateral flow assay having a substrate configured to form a blood spot thereon upon application of a sample of whole blood onto said substrate, at least one light source, a detector arranged to detect light reflected from said blood spot and to determine the reflectance and/or size of said blood spot, and a processor configured to correlate the reflectance and/or the size of the blood spot to a hemoglobin concentration of said sample based on stored values of reflectance and/or size obtained from known hemoglobin concentrations, and to calculate the hematocrit level based on said hemoglobin concentration.

One aspect relates to a lateral flow assay device for determining the concentration of plasma calprotectin in a whole blood sample, wherein said device comprises a substrate configured to form a blood spot thereon upon application of a sample of whole blood onto the substrate, a conjugate pad with anti-calprotectin antibodies conjugated to a marker, a membrane with at least one test line of immobilized anti-calprotectin antibodies, and an absorbent pad.

Another aspect relates to a lateral flow assay device for determining the concentration of cystatin C in a whole blood sample, wherein said device comprises a substrate configured to form a blood spot thereon upon application of a sample of whole blood onto the substrate, a conjugate pad with anti-cystatin C antibodies conjugated to a marker, a membrane with at least one test line of immobilized anti-cystatin C antibodies, and an absorbent pad.

Yet another aspect relates to a lateral flow assay device for determining the concentration of ferritin in a whole blood sample, wherein said device comprises a substrate configured to form a blood spot thereon upon application of a sample of whole blood onto the substrate, a conjugate pad with anti-ferritin antibodies conjugated to a marker, a membrane with at least one test line of immobilized anti-ferritin antibodies, and an absorbent pad.

Another aspect relates to a lateral flow assay device for determining the concentration of plasma procalcitonin in a whole blood sample, wherein said device comprises a substrate configured to form a blood spot thereon upon application of a sample of whole blood onto the substrate, a conjugate pad with anti-procalcitonin antibodies conjugated to a marker, a membrane with at least one test line of immobilized anti-procalcitonin antibodies, and an absorbent pad.

Another aspect relates to a lateral flow assay device for determining the concentration of C-reactive protein (CRP) in a whole blood sample, wherein said device comprises a substrate configured to form a blood spot thereon upon application of a sample of whole blood onto the substrate, a conjugate pad with anti-CRP antibodies conjugated to a marker, a membrane with at least one test line of immobilized anti-CRP antibodies, and an absorbent pad.

Another aspect relates to a lateral flow assay device for determining the concentration of anti-CCP antibodies in a whole blood sample, wherein said device comprises a substrate configured to form a blood spot thereon upon application of a sample of whole blood onto the substrate, a conjugate pad with one or more cyclic citrullinated peptides (CCP) conjugated to a marker, a membrane with at least one test line of immobilized antibodies, and an absorbent pad.

In an embodiment of a lateral flow assay device according to any one of the aspects above, said substrate is arranged in fluid connection with the conjugate pad of said lateral flow assay, and wherein said substrate is a glass fiber-based filter. Where a glass fiber-based substrate is used, the reflectance is preferably measured at a wavelength in the interval 650 nm-1000 nm.

In another embodiment of a lateral flow assay device according to any one of the aspects above, said substrate is arranged parallel to and not in fluid connection with the conjugate pad of said lateral flow assay, and wherein said substrate is chosen from a glass fiber-based filter, a cellulose-based filter, and a substrate having an impermeable surface. Where a cellulose-based substrate is used, the reflectance is preferably measured at a wavelength in the interval 550 nm-630 nm.

Another embodiment is a device for receiving a lateral flow assay device according to any one of the embodiments above, comprising at least a light source, a detector arranged to detect reflected light and/or to determine the size of a blood spot formed on a substrate of said lateral flow device, and a processor configured to correlated the reflectance and/or size of the blood spot to a hematocrit level and to use this hematocrit level when calculating the concentration of an analyte in a sample of whole blood.

Other embodiments relate to a processor configured for converting reflectance values and/or size of the blood spot into a hematocrit level based on stored values of reflectance and/or size obtained for known hematocrit levels. Further, said processor is configured for taking the hematocrit level of a sample into account when calculating the value of another analyte present in plasma, in a setting where a sample of whole blood has been subjected to analysis. Said processor is provided using any combination of one or more of a suitable central processing unit (CPU), multiprocessor, microcontroller, digital signal processor (DSP), application specific integrated circuit etc., capable of executing software instructions stored in a memory, which can thus be a computer program product. The processor can be configured to execute any one of the methods disclosed herein, for example the methods defined in the claims attached hereto.

This memory can be any combination of random access memory (RAM) and read only memory (ROM). The memory also comprises persistent storage, which, for example, can be any single one or combination of magnetic memory, optical memory, solid-state memory or even remotely mounted memory.

A data memory is also provided for reading and/or storing data during execution of software instructions in the processor. This data memory can be any combination of random access memory (RAM) and read only memory (ROM).

A device and/or a system for lateral flow assay as disclosed herein can further comprise an I/O interface for communicating with other external entities. Optionally, the I/O interface also includes a user interface.

Other components of are omitted in order not to obscure the concepts presented herein.

In the above aspects and embodiments, applying the whole blood sample to a substrate comprises applying the sample, e.g. a drop of blood, onto a substantially flat fibrous structure, such as a chromatography paper, cellulose-based filter or a glass fiber-based filter. The size of the substrate, e.g. a paper or filter, is made proportionate to the sample size, so that for example a 25 μl drop of blood forms a substantially round spot without the edges of the spot reaching the edges of the paper or filter. In the examples, strips of filter paper 5 mm wide were initially tested, but in the majority of the examples, squares of 15 mm×15 mm were used and the sample (25 μl) was applied approximately to the middle of each square. For a larger sample volume, the size of the paper or filter will be correspondingly larger. The sample volume can be chosen by a skilled person in the art, and can be, for example, 10 μl, 20 μl, 25 μl, 50 μl, 100 μl depending on the assay in question, e.g. the analyte to be determined, the physiological concentration of the analyte, and the sensitivity of the assay.

It is surprising that the above mentioned analytes, present in plasma, can be accurately measured in a whole blood sample applied to a lateral flow test, and the methods and devices disclosed herein offer many benefits in the field of diagnostics, in particular in the Point-of-Care setting.

One important advantage of the methods disclosed herein is that they do not rely on the use of any reagents for the determination of Hct and/or Hb. This not only simplifies the analysis, it also reduces the cost, as well as the risk, considering that the most frequently used reagents in Hb measurements are potassium ferricyanide and potassium cyanide, two highly toxic chemicals.

The elimination of reagents such as potassium ferricyanide and potassium cyanide has an additional advantage in that the lateral flow assay device becomes more stable during storage, and can be approved for longer shelf-life as these and other reagents are prone to absorb moisture and/or react with other components of the assay.

Another advantage is the rapid response time, making it possible to have a result already during the visit to the physician or the clinic, when screening blood donors etc. The rapid response time also makes it possible to integrate the determination of Hb and Hct into assays determining the concentration of other analytes in plasma, and to correct for variations in Hct. The present inventors have shown that this significantly improves the precision of the analyte measurement, compared to measurements where an average Hct (average for the total population or average for the gender of the patient) is used. An integrated measurement of Hct is significantly more reliable than methods where the Hct is estimated based on the patient's gender, age and possibly health status.

Another advantage is that the method and devices are substrate-based, e.g. paper-based, and thus no separate cuvette is needed, which in turn makes the method—as well as devices using said method—simpler and less costly. Compared to the use of cuvettes comprising lyophilized reagents, the assay methods and devices are more stable, and less sensitive to variations in the ambient humidity.

Yet another advantage is that the immediate measurement, in contrast to prior art methods that require lengthy drying of the samples, helps to avoid mix-ups of samples, avoids long waiting time for the patient, or when the method is automatized, allows higher through-put.

In addition to being fast and simple, the method disclosed herein is surprisingly accurate. Based on the experience obtained by the inventors so far, the accuracy is within ±7 g/l Hb or about ±2% Hct.

Another advantage is that camera sensor technology, or alternatively any other detector arrangement suitable for the stated purpose, is relatively cheap and easily available, making it economical to build analysis devices utilizing the inventive concept. The method is also well suited for automation, which is another important advantage.

The methods disclosed herein can also be integrated in or performed simultaneously or sequentially with existing and future clinical analysis methods, as a separate step in the sample preparation, as an initial step in the handling of the sample in the assay device, for example, when a sample is applied to a so-called blood filter for the separation of red blood cells from plasma. It is in this context a significant advantage that the wavelength interval (range), in particular the interval 650 nm to 1000 nm, is equally applicable to cellulose-based and glass fiber-based substrates. It is also an advantage that the determination of Hct is performed without hemolysing the blood, as this allows the integration of the method into assays where hemolysis is undesired, either because the discoloration of the plasma would interfere with the reading of the result, or where hemolysis releases substances which interfere with the analyte or reagents, compromising the assay.

### EXAMPLES

### Example 1. Optical Hemoglobin Measurements

### Materials and Methods

### Optical Set-Up, Software and Methods

A Sony A7 II mirrorless consumer camera housing with 70 mm f/2.8 DG Macro Art lens (Sony Co., Japan) was used to take the images. The camera has a full format CMOS sensor and provides 24 MP images.

A light box was made by using a white Styrofoam® transport box and household warm LED spots (3000 K) were bought and installed in the box. The lighting was adapted to the type of image. When sample spots on paper were investigated, light from many sides in the box was used, while when a sample in capillaries were investigated, the light sources were placed below the object, illuminating the objects from underneath, to avoid reflections. Also, a white balance card (Porta-Brace Inc., USA) was used as a table for the objects to be imaged. Tissue paper/paper towels were used to diffuse the light when necessary.

The camera was placed in exactly the same position in each experiment, but with different camera settings, different distances between sensor and object, and different focus settings (manually adjusted to the distance in question). All images were taken in RAW format (Sony RAW). Since the RAW format color image is an array of 3 colors, and there is a Bayer color filter, the green array contains information in 50% of the image pixels, while the red and blue channels contain information in 25% of the image pixels.

The images were analyzed using the red array (R), and since it was difficult to find a good way to read the RAW images directly, the RAW images were converted to TIF format. This means that demosaicing took place to get color intensity values in all pixels for all color channels. In addition, a gamma correction could have taken place in the conversion from RAW to TIF.

Tagged Image File Format, abbreviated TIFF or TIF, is a computer file format for storing raster graphics images. The ability to store image data in a lossless format makes a TIFF file a useful image archive, because, unlike standard JPEG files, a TIFF file using lossless compression (or none) may be edited and re-saved without losing image quality.

Sony Imaging Edge software (Sony Co., Japan) was used to open ARW images and convert them to TIF. The images were then saved as Wide Gamut RGB (for viewing) and 16 bit (although the image sensor has a dynamic range of 14 bit). A compression was also done in some cases to keep the images to a reasonable size. In total, the original signals in the RAW images were transformed in the TIF conversion, and reading the raw images was postponed for future studies.

As the RAW images are not images as such, only partially filled matrices/arrays with numbers, using RAW images did not allow the performing of ordinary image analysis, such as clustering etc. For each pixel there is only one color value in the RAW image, 50% of the pixels contains a green signal, 25% of the pixels contains a red signal and 25% of the pixels contains a blue signal. However, since there are so many pixels in an image, the algorithms assume that a pixel takes the color intensities of its neighbours.

### Blood Samples

Ten whole blood non gel heparin samples were collected on two occasions, on Aug. 15 and Aug. 27, 2018. Different donors were used each time. The samples were assigned a hemoglobin concentration by taking the average of a Hemocue 201 (Hemocue AB, Sweden) measurement and a Drabkin measurement. (The Drabkin measurement is a standardized spectrophotometric method, wherein blood is diluted in a solution containing potassium ferricyanide and potassium cyanide. Potassium ferricyanide oxidizes the iron in heme to the ferric state to form methemoglobin, which is converted to hemiglobincyanide (HiCN) by potassium cyanide. HiCN is a stable colored product, which in solution has an absorbance maximum at 540 nm and strictly obeys Beer-Lambert's law. Absorbance of the diluted sample at 540 nm is compared with absorbance at the same wavelength of a standard HiCN solution whose equivalent hemoglobin concentration is known. Reagents were obtained from Randox Laboratories Ltd., UK).

From the ten samples collected on Aug. 15, 2018, a set of 8 calibrators were made from one of the samples by dilution with corresponding plasma and spiking with removed red blood cells. In addition, the samples were diluted and mixed, and in total 22 samples were used. The initial idea was to make a calibration curve from the 8 calibrators and then use the 22 samples to evaluate the 22 samples versus the assigned concentration. However, this strategy was abandoned, and instead the calibrators and the samples were all used for modelling and validation of a model for hemoglobin quantification. A cross validation with 2 splits was used and resulting model was used to evaluate concentration of all the samples. This is not a perfect strategy, but the number of samples was judged too low to allow for a separate hold-out set in order to evaluate the model with samples that have not been involved in model building.

From the 10 samples collected on Aug. 27, 2018, a total of 36 samples were prepared by mixing and diluting the samples with corresponding plasma. All samples were used for modelling and validation purposes. As described above, a 2 split cross validation was done and no hold-out set was used.

### 1.1 Optical Measurements on Filter Paper

Samples of filter paper (Whatman® 2668 cellulose chromatography paper, Merck KGaA, Germany) were cut by hand. An optical set-up as described above was used, with the camera settings ISO 160, shutter speed 1/100 and aperture 10.

100 μl blood was pipetted onto pieces of filter paper and an image taken immediately after application, estimated time less than 1 min after application for each sample.

The images were cropped manually and converted to TIF. The crops were 2000×2000 pixels and the blood spots were not always centered in the images due to the need to crop away written numbering on the paper. The size of the blood spots was found to be correlated to the hemoglobin concentration.

The images were converted to grayscale and thresholded by grayscale intensity of 0.7. Then the number of white pixels in each binary image was counted to determine the area covered by blood. Thereafter, statistics from each blood spot was collected for each color channel as well as mean and median background signal for each color channel. Background corrected signals were generated by multiplying the blood spot signals by the background signals. Correlations of the generated statistics and the number of pixels belonging to blood spots versus the assigned concentration showed that the median blood spot signals were highly correlated to assigned concentration. Also the number of blood pixels in the images seems fairly well correlated to assigned concentration. Interestingly, the red channel background is also correlated to concentration, probably meaning that some blood pixels have contaminated the background, and that the filter value used could be improved. Samples<8 g/dl were removed (in this case 1 sample).

The median corrected red, green and blue channels and the feature representing the number of blood pixels were used to make multivariate models. The final dataset was standardized, and PC transformed. Modelling used all 4 PCA components, representing the median corrected color channels. MLR and SVM with linear kernel were performed using R and package caret. Concentration was used as weight in the modelling. The models were built by doing two-fold cross validation, and all the samples were predicted by the final model. No hold-out set was used. The average of the MLR and SVM model is presented. Two samples had bias>1 g/dl, and the most problematic samples seems to be the high samples around 15 g/dl

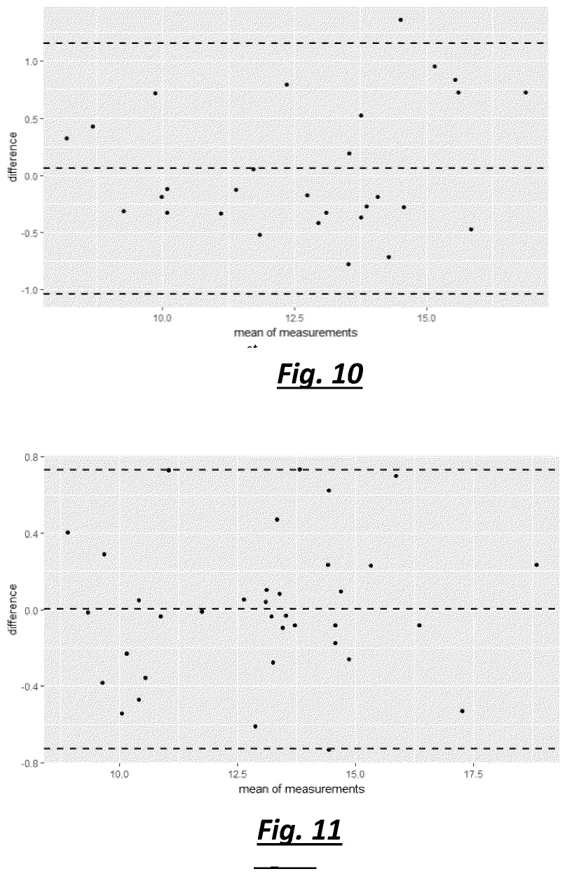

The results are shown as a Bland Altman plot in FIG. 10.

Experiments using different paper qualities were also performed. 50 μl whole blood was applied onto hand cut pieces of Whatman® 17 CHR cellulose chromatography paper. In this experiment, the light setting was different from the capillary experiments as the LEDs were placed on all sides of the box instead of only using backlight. The camera settings were ISO 160, shutter speed 1/100 and aperture 10.

Color temperature was set to 2800 K as in all the other experiments. The results are shown as a Bland Altman plot in FIG. 11.

### 1.2 Comparative Example: Optical Measurements in a 20 μl VITREX® Capillary

Samples of whole blood were collected in 20 μl VITREX® glass capillaries (Vitrex Medical A/S, Denmark) and photographed in a light box against the background of a white balance card and using the camera as presented above, using the camera settings ISO 250, shutter speed 1/80 and aperture 10. The images were inclination corrected and cropped using Sony Imaging Edge.

The images were converted to grayscale and pixels were segmented according to grayscale intensity<0.7 belonging to the capillary, while the rest belongs to the background. The choice of 0.7 was based on the fact that most pixels in the background region were around 0.85 in intensity. Examples of segmented Boolean image indicated that this segmentation is fairly reasonable. However, more sophisticated segmentation/cropping could be done. The choice of cut-off seems to affect the results slightly. 0.7 seems most appropriate after attempting 0.6, 0.7 and 0.8. Also, a background signal<1 indicates a background different from white. Since the background is a white balance card, one could have adjusted white balance, but this was not done. All the images are taken in the same light box under the same light conditions.

Attempts to adjust for background signal was done, by attempting to divide and multiply the background by the mean and median signals in the red region. Correlations improved slightly, but probably not significantly. A proper background correction is possibly important. Looking at correlations, it seems that median signals corrected by median background are the best choice of predictors.

Before modelling, the 3 predictors were PCA transformed after standardization. This worked best with an MLR model with all 3 PCA components. The model was weighted by 1/Assigned concentration. Other models were attempted, such as SVM with linear kernel, but did not improve results. An average of MLR and SVM was used and gave the Bland Altman plot shown in FIG. 11, demonstrating that several samples had biases>1 g/dl versus the assigned values.

Experiments using a 40 μl VITREX® capillary were also performed, providing comparable results (results not shown). Similarly, additional experiments using capillaries were performed, in which the capillaries were laid on foam supports glued to the white balance card in order to separate the capillary from the white balance card or placed between two pieces of black plastic. Various camera settings were also tried, changing the exposure of the capillaries.

### 1.3 Comparative Example: Optical Measurements in a Rectangular Capillary

Samples of whole blood were collected in rectangular glass capillaries (Hilgenberg GmbH, Germany) and photographed in a light box against the background of a white balance card and using the camera as presented above, using the camera settings ISO 200, shutter speed 1/80 and aperture 10.

The images were cropped to 3000×500 pixels and saved as wide gamut 16 bit uncompressed TIF. Background corrected signals were generated by dividing the blood signals by the background signals. Correlations of the generated statistics and the number of pixels belonging to blood versus the assigned concentration showed that the median blood signals were moderately correlated to assigned concentration Samples<8 g/dl were removed (in this case 1 sample). Six samples had bias>1 g/dl, and the most problematic samples seem to be the high samples around 15 g/dl, and in general the predicted values are off in comparison to the assigned values.

The results showed that optical hemoglobin quantification based on digital image color analysis is feasible, and that—in the present experiments—the analysis performed better when the samples were applied to a paper medium compared to glass capillaries.

### Example 2. Optical Hb Measurements on Filter Paper

### Materials and Methods

### Optical Set-Up, Software and Methods

A Sony A7 II mirrorless consumer camera housing with 70 mm f/2.8 DG Macro Art lens (Sony Co., Japan) was set up as described in relation to Example 1.

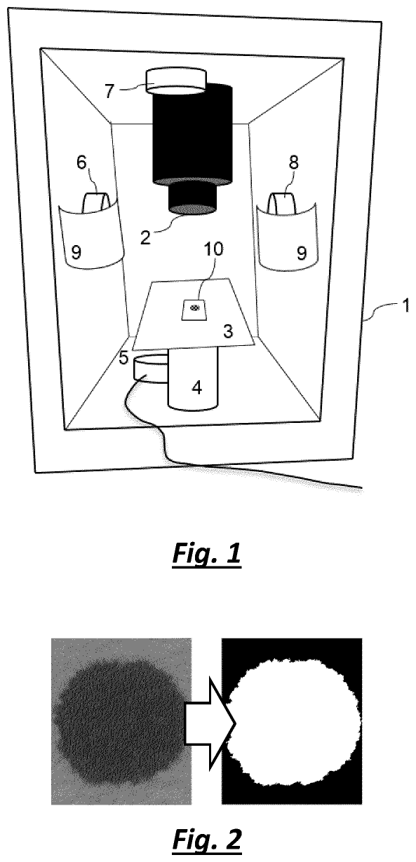

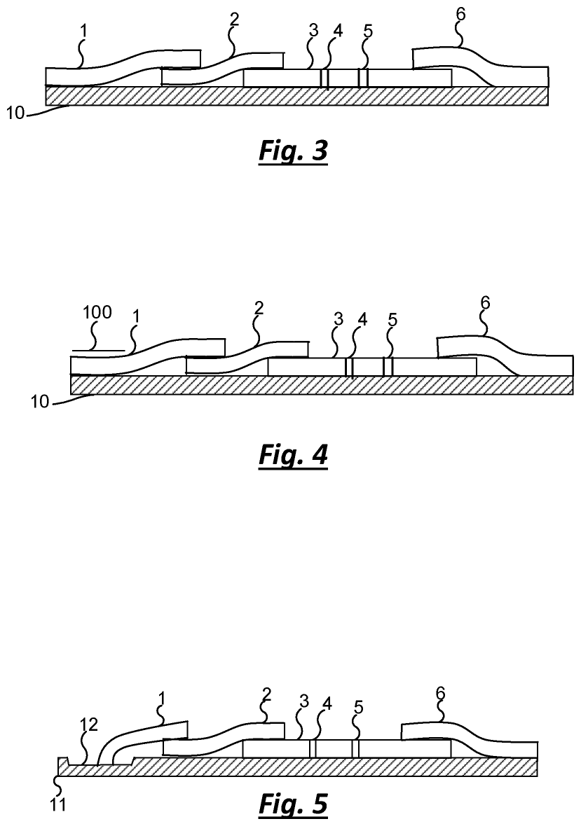

The experimental set-up is shown schematically in FIG. 1. In the drawing, the front of the box 1 has been removed, exposing a camera 2, a “table” or sample holder 3 for holding the samples, an adjustable support 4 for positioning the “table” at different distances from the lens of the camera 2. Four light sources 5, 6, 7 and 8 were positioned inside the box, and could be individually turned on and off. The light could be diffused by placing a paper screen 9 in front of each one of the light sources. The samples 10 were placed on the “table” directly under the lens of the camera 2.

In each experiment, the camera was placed in exactly the same place, but with different camera settings, different distances between sensor and object, and different focus settings (manually adjusted to the distance in question). As disclosed for Example 1, also here all images were taken in ARW format (the Sony Alpha RAW format) and analysed using the red array (R). Similarly as in Example 1, Sony Imaging Edge software (Sony Co., Japan) was used to open RAW images and to convert them to TIF. The images were then saved as Wide Gamut RGB (for viewing) and 16 bit (although the image sensor has a dynamic range of 14 bit). A compression was also done in some cases to keep the images to a reasonable size.

### Blood Samples

For the initial experiments, ten whole blood non-gel heparin samples were collected on two occasions. Different donors were used each time. The samples were assigned a hemoglobin concentration by taking the average of a Hemocue 201 (Hemocue AB, Sweden) measurement and a Drabkin's measurement (reagents and manual from Randox Laboratories Ltd., UK).

From the ten samples collected on the earlier of the two occasions, a set of 8 calibrators were made from one of the samples by dilution with corresponding plasma and spiking with corresponding plasma removed red blood cells. In addition, the samples were diluted and mixed, and in total 22 samples were used. The calibrators and the samples were used for modelling and validation of a model for Hb quantification. A cross validation with 2 splits was used and resulting model was used to evaluate concentration of all the samples.

From the 10 samples collected on the later of the two occasions, a total of 36 samples were prepared by mixing and diluting the samples with corresponding plasma. All samples were used for modelling and validation purposes. As described above, a 2 split cross validation was performed. No hold-out set was used.

Samples of filter paper (Whatman® 2668 cellulose chromatography paper, Merck KGaA, Germany) were cut by hand. An optical set-up as described above was used, with the camera settings ISO 160, shutter speed 1/100 and aperture 10.

A sample of blood was pipetted onto pieces of filter paper and an image taken immediately after application, estimated time less than 1 min after application for each sample. The sample size was 100 μl blood.

The images were cropped manually and converted to TIF. The crops were 2000×2000 pixels and the blood spots were not always centred in the images due to the need to crop away written numbering on the paper. The size of the blood spots was found to be correlated to the hemoglobin concentration.

The images were converted to grayscale and thresholded by grayscale intensity of 0.7. Then the number of white pixels in each binary image was counted to determine the area covered by blood. Thereafter, statistics from each blood spot was collected for each colour channel as well as mean and median background signal for each colour channel. Background corrected signals were generated by multiplying the blood spot signals by the background signals. Correlations of the generated statistics and the number of pixels belonging to blood spots versus the assigned concentration showed that the median blood spot signals were highly correlated to assigned concentration. The number of blood pixels in the images seemed well correlated to assigned concentration. Interestingly, the red channel background is also correlated to concentration, probably meaning that some blood pixels have contaminated the background, and that the filter value used could be improved. Samples<8 g/dl were removed (in this case 1 sample).

The median corrected red, green and blue channels and the feature representing the number of blood pixels were used to make multivariate models. The final dataset was standardized, and PC transformed. Modelling used all 4 PCA components, representing the median corrected colour channels. MLR and SVM with linear kernel was performed using R and package caret. Concentration was used as weight in the modelling. The models were built by doing 2-fold cross validation, and all the samples were predicted by the final model. No hold-out set was used. The average of the MLR and SVM model is presented. Two samples had bias>1 g/dl, and the most problematic samples seemed to be the high samples around 15 g/dl.

Experiments using different paper qualities were also performed. 50 ul whole blood was applied onto hand cut pieces of Whatman® 17 CHR cellulose chromatography paper. In this experiment, the light setting was different from the capillary experiments as the LEDs were placed on all sides of the box instead of only using backlight. The camera settings were ISO 160, shutter speed 1/100 and aperture 10. Colour temperature was set to 2800 K as in all the other experiments.

### Example 3. Optical Measurement of Hb on Paper—Large Scale Trials

Fresh lithium heparin venous blood from voluntary donors is collected. A total of 60 samples, originating from 20 donors were prepared as follows: The 20 original donors donated two vials of lithium heparin blood. One vial was centrifuged to obtain plasma which was used to dilute blood from the same donor. Part of the 20 original samples were diluted to approximately 75% of the original concentration using corresponding plasma, giving in total 20 samples. Part of the original samples were mixed, and some diluted to approximately 50% by corresponding plasma to give a total of 60 samples with Hb concentrations ranging from 80 to 180 g/L. The Hb concentration of each sample was assigned using three different methods:

Analysing with a HemoCue 201+ device (Hemocue AB, Ängelholm, Sweden)

Manual Drabkin's method (reagents and instructions from Randox Laboratories Ltd., Crumlin, UK)

CO Oximetry method, for example as described by Attia et al., Determination of Human Hemoglobin Derivatives, Hemoglobin, 2015; 39(5): 371-374.

The results showed that 47 of the total 60 samples had a CV<3.8% when all three values were compared. The mean of the three methods was assigned to each sample.

Several experiments were performed, all using 25 μl whole blood which was added to Whatman™ CHR 17 chromatography paper. A selection of bandpass filters were used: 543.5 nm, 590 nm, 660 nm, and 940 nm.

The Sony A7 II colour 24 MP camera was used with a Sigma Art 70 mm f/2.8 lens, and the images collected in Sony ARW format (RAW format), exposure time 1/50 sec, ISO 100, aperture 13.

The set-up was closely similar to that described for Example 1, see FIG. 1. A 16 mm lens extender was mounted on the Sony A7 II camera, and then the Sigma Art 70 mm f/2.8 lens. On the lens, a sun protection was mounted and extended about 2 cm using black tape. The box was covered with black aluminium foil to keep light conditions stable during the experiment. Tissue paper was used to diffuse the light and avoid shadowing. 3 small LED spotlights of 100 lumen each and a colour temperature of 2900 K were used to illuminate the samples. The light source was adapted to the spectral region of interest. If operating in the UV-VIS region, white LED light is used, while if operating in the NIR region, halogen light is used.

A sample of blood was pipetted onto pieces of filter paper and an image taken immediately after application, estimated time less than 1 min after application for each sample. The sample size was 100 μl blood.

All images were taken in duplicate, and background images (without blood) were taken at the beginning and end of each experiment. The blood spot was identified by analysing the contrast between background and blood, and a mask created for each spot in order to focus on the pixels of each spot. See FIG. 2.

After imaging the blood spots using the Sony colour camera set-up, the samples were immediately transferred to a monochrome camera set-up, using a Pixelink PL-D795MU-5MP monochrome camera (5 MP, 2/3 sensor) and a VZM 100i video lens (Edmund Optics). A manual filter wheel was mounted at the end of the lens, allowing the alternation between different filters. The images were taken as 8 bit TIF without gamma correction. The aperture of the lens was fully open and the magnification was set at 0.75×, exposure time 70 ms. First, a 590 nm filter was used and the sample illuminated with two 50 W LED spots, having a colour temperature of about 2700 K. Again, duplicate images were taken for each sample, and blank/background images were taken at several time points. The sample and camera were enclosed with blackout material (Thorlabs) to prevent changing external light conditions from influencing the images.

The experiment was repeated with the 543.5 nm filter. When changing to the 660 nm filter, the light source was changed to a 50 W (350 lumen) LED and the experiments were repeated as above.

After using various image processing techniques, the final signals (R, G and B colour channels) were extracted from all images and correlated to the corresponding hemoglobin concentration. The best results, in terms of signal correlation to Hb concentration, were achieved with gamma correction and background division (image/background), and Pearson correlations of about 0.90 was achieved for all channels. N.B. a Pearson correlation is a number between −1 and 1 that indicates the extent to which two variables are linearly related.

Before building a final model, all data was used in building a linear model for 600 nm and a second-order polynomial model for 940 nm. To build the models, the raw data was split into a 60% training set and a 40% test set. The models were built by using 3-fold cross validation in R's package caret (classification and regression training). The test set was predicted for both models and averaged. A Bland-Altman analysis was performed. The process was repeated 3 times with different splits of the dataset to obtain average limits of agreement over 3 runs. The results showed that the average 95% limits of agreement of the three different splits of validation and test set on average gave Bland-Altman 95% limits of agreement of −6 and 6 g/l. On average, only 1.67 samples had higher bias than 5%.

It was seen that building two models, one for each wavelength, predicting the hemoglobin values from each of the models and then averaging the two results gave fairly accurate hemoglobin estimates. In 95% of the cases, the predicted values were within ±0.7 g/dl from the assigned values, which is very close to a typical bias specification for a hemoglobin POC device.

To summarize, the method can accurately quantify hemoglobin in a fresh whole blood sample by adding a drop of blood onto a high-quality filter paper and imaging the blood spot using a camera, a lens, one or more proper bandpass filters or narrow banded LEDs. In conclusion, these early experiments indicate that 660 nm is a good single indicator for Hb concentration, and that a combination of 660 nm and 940 nm is a good predictor.

### Example 4. Experiment Evaluating Different Filter Media Using 6 Samples (1 Donor)

Blood samples: One donor having Hb 168 g/l volunteered for this experiment. From this donor's blood, six samples were prepared, having the concentrations of 67 g/l, 100 g/l, 136 g/l, 168 g/l, 199 g/l and 216 g/l.

Substrate/Filter medium: The different filter media evaluated in this experiment are presented in Table 1.

TABLE 1
Substrates tested

| Brand | Manufacturer | Type | Thickness | | Flow rate |
|---|---|---|---|---|---|
| Whatman ™ 17 | Thermo Fisher | Cellulose based | 0.92 | mm | 19 cm/30 min |
| CHR | Scientific, Inc. | filter paper | | | |
| Whatman ™ | Thermo Fisher | Cellulose based | 0.9 | mm | |
| Grade 2668 Chr | Scientific, Inc. | filter paper | | | |
| GF/DVA | GE Healthcare | Bound glass | 758 | μm | 4 cm/44 s |
| | | fiber | | | |
| VF2 | GE Healthcare | Bound glass | 785 | μm | 4 cm/23.8 s |
| | | fiber | | | |

Optical filters: The optical filters and the corresponding exposure times evaluated in this experiment are presented in Table 2.

TABLE 2
Optical filters and exposure times

| Wavelength (nm) | Exposure time (ms) |
|---|---|
| 543.5 | 500 |
| 590 | 500 |
| 660 | 500 |
| 780 | 800 |
| 800 | 1200 |
| 940 | 3000 |

The experiments were performed substantially as described above, and experimental set-up was substantially the same, corresponding to that shown in FIG. 1, and the same four filter media and the six different optical filters were used. In this example, however, the exposure times were optimized for each filter as shown in Table 2. The images were saved as 16 bit TIF.

25 μl samples of blood were prepared, and the Hb and Hct values determined by using a Hemocue 201 device (Hemocue AB, Sweden) and a Drabkin's measurement (reagents and manual from Randox Laboratories Ltd., UK) and a Haematokrit 200 centrifuge (Andreas Hettich GmbH & Co.KG, Tuttlingen, Germany). The samples were applied to 15 mm×15 mm squares of the tested substrates. The blood spots were imaged and the images analysed to first identify the constituting pixels (see FIG. 2). The images were corrected for unevenness in the illumination profile by comparing to images of substrates without any blood applied on them.

The background was corrected by also looking at the intensity of the “paper pixels” adjacent to the defined blood spot. Different parameters were then extracted from the histograms of pixel intensities: mean, median, mode and mode-of-fit. The expression “mode-of-fit” is here intended to represent the most common pixel intensity in the defined blood spot. However, as the histogram over pixel intensities was a bit noisy, “mode” alone was not a viable option. Instead a smooth continuous curve was fitted to the histogram peak from which the most common pixel intensity was extracted.

Currently the median and the mode-of-fit parameter are considered as the most promising, as it represents the peak value of the fit to the histogram and disregards noise.

### Example 5. Experiment Using 18 Samples (3 Donors)

In another round of measurements, blood was obtained from three donors, and diluted to a total of 18 samples with different Hb ranging from 50 to 166 g/l. The experimental setting was the same, corresponding to that shown in FIG. 1, and the same four filter media and the six different optical filters were used.

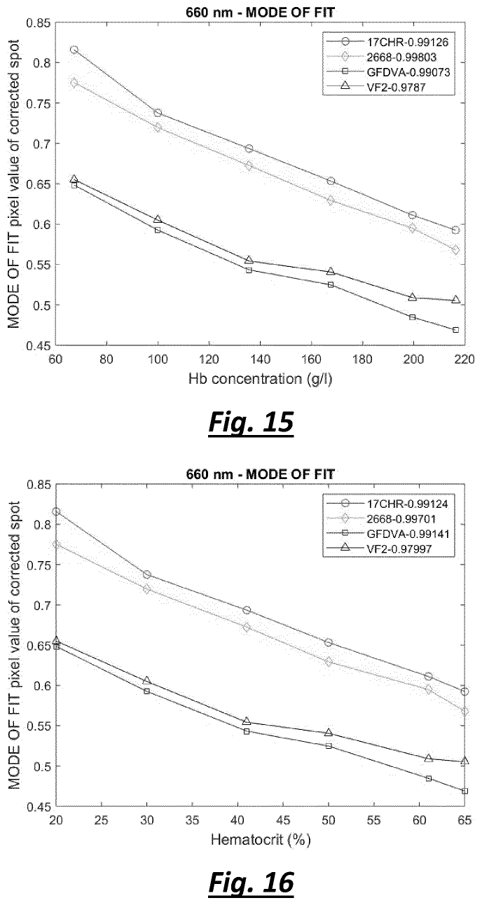

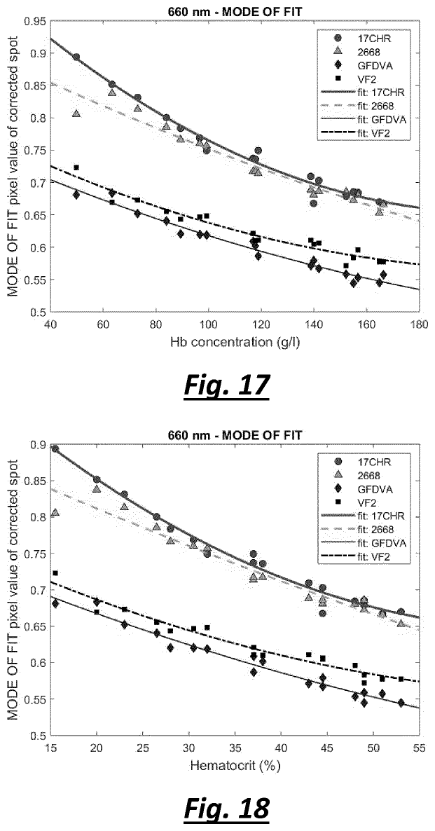

The results shown in FIGS. 15 and 17 prove that good correlation was obtained for all 4 filter media using 660 nm and when extracting mode-of-fit from the pixel values in the corrected spot in each image. When correlating the measurements to hematocrit, equally good results were obtained, see FIGS. 16 and 18.

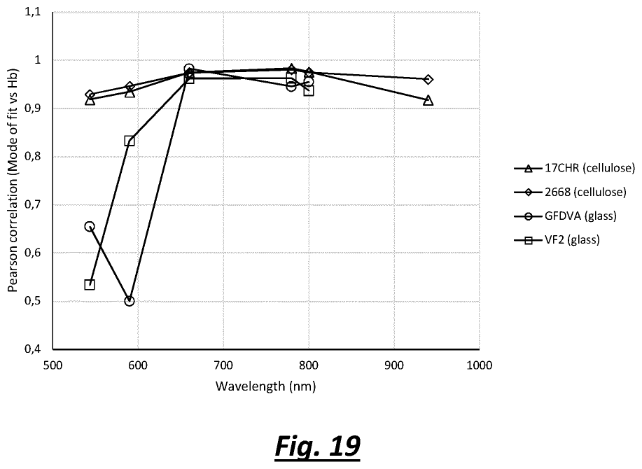

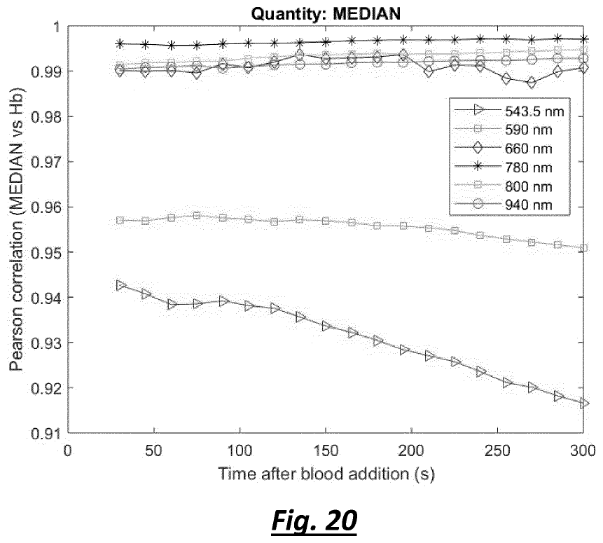

FIG. 19 shows the Pearson correlation between mode-of-fit and Hb for the four different filter media at six different wavelengths. The graph shows that all four filter media can be used in a wavelength interval of 660 nm to at least 800 nm. The corresponding correlation plot for the hematocrit value is essentially identical and is therefore omitted.

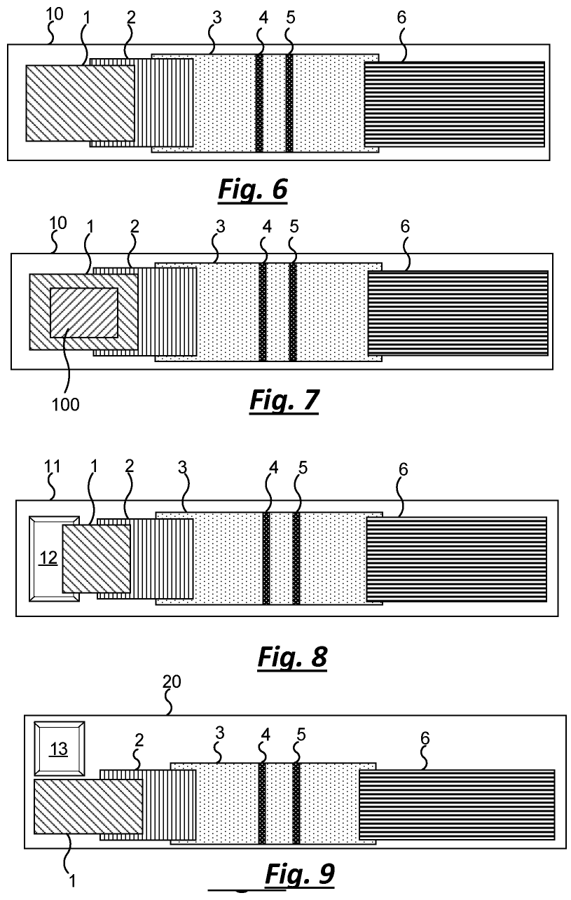

It can be seen that the glass fiber-based filter media performed poorly at the lower wavelengths 543.5 and 590 nm but equalled the performance of the cellulose-based filter media at 660 nm, 780 nm and 800 nm. It is contemplated that a cellulose-based filter media is chosen as the substrate in applications where hemolysis is not a concern, for example applications where the determination of Hct volume fraction is done separately from, e.g. in parallel with another measurement of an analyte present in plasma. Conversely, where the determination of Hct is performed in line with one or more analysis of other analytes, it is preferred that hemolysis is minimized or entirely prevented. This applies in particular to lateral flow assays. When a quantitative determination of an analyte present in plasma is performed downstream in a lateral flow assay, it is preferred that a non-hemolysing sample pad or substrate is used, on which the Hct is determined, before the plasma is led further along the test strip. These alternative embodiments are illustrated e.g. in FIG. 6 and FIG. 9. In FIG. 6, item 1 represents a sample pad in fluid flow connection with the remaining lateral flow assay strip or flow path. In such an embodiment, the substrate or sample pad 1 is preferably a glass fiber-based filter or similar non-hemolysing substrate.

In the embodiment schematically shown in FIG. 9, item 13 represents a sample addition point which is not in fluid flow connection with the lateral flow assay. Here, the sample is added in parallel to the sample pad 1 and to the separate sample addition site 13, which can be a recess, an impermeable or semipermeable substrate, a membrane or a filter paper, preferably a cellulose-based filter paper.

A set of measurements were performed to evaluate if the capability to determine Hb and Hct is preserved over time. The Whatman™ 17 CHR chromatography paper was used in this experiment. 12 different samples were analysed at 20 time points (15-300 seconds, with 15 second intervals). The results are shown in FIG. 20 which shows the Pearson correlation (median v. Hb) as a function of time for six different optical filters. It is clearly seen that for the higher wavelengths, at least 780 nm, 800 nm and 940 nm, the sample is very stable with regard to the measured parameters. The measurement series for 660 nm is less accurate, but it is seen that on average, also the 660 nm measurements indicate a good correlation over time.

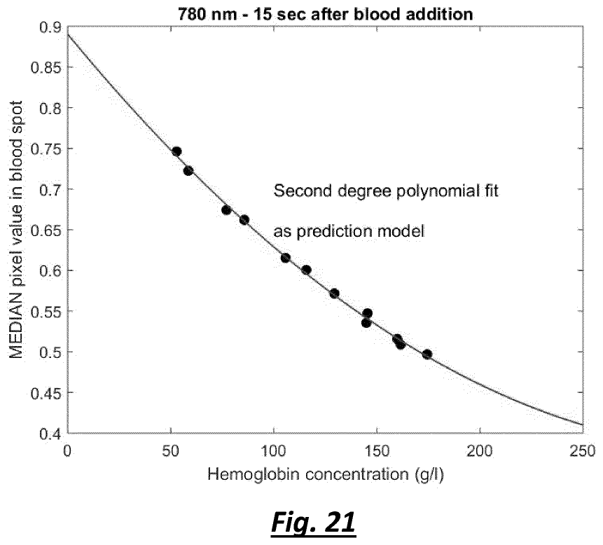

The inventors built a prediction model by fitting a second-degree polynominal to the Hb results obtained at 15 seconds after addition of the blood sample, determined at 780 nm. See FIG. 21.

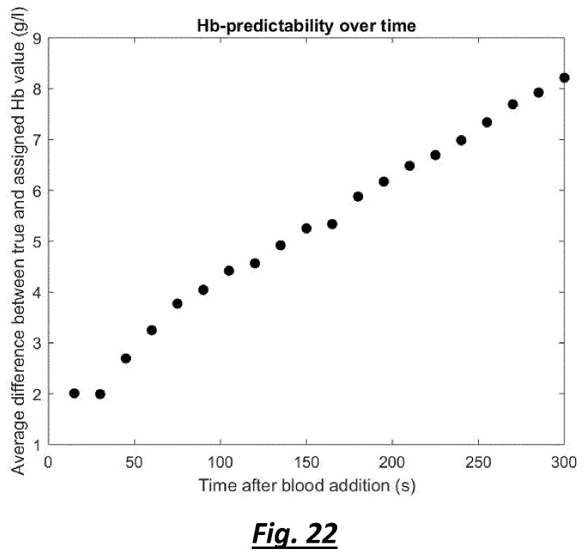

This model was then used to predict the Hb at the remaining 19 timepoints (30, 45, 60, 75, 90, 105, 120, 135, 150, 165, 190, 205, 220, 235, 250, 265, 280, 295 and 300 s). The results are shown in FIG. 22 where the average difference between true and assigned Hb value (g/l) is shown as a function of time. It can be seen that based on this prediction model, the prediction power deteriorates with time. Based on FIG. 20, one would contemplate that the measurement should be performed within 1 to 60 seconds, from application of the sample. However, one can assume that the method is stable for at least the first 300 seconds, at least when using the wavelengths 780, 800 and 940 nm.

### Example 6. Ferritin Quantification Assay Based on Lateral Flow

### Materials and Methods

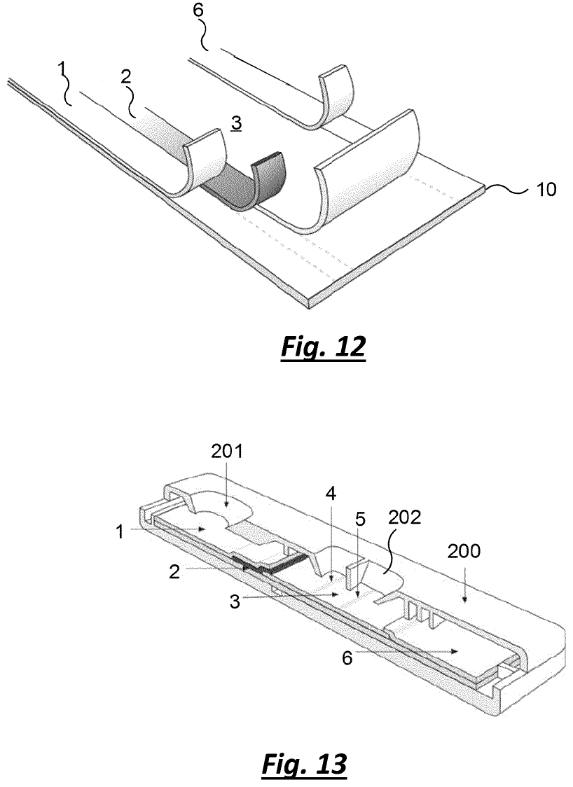

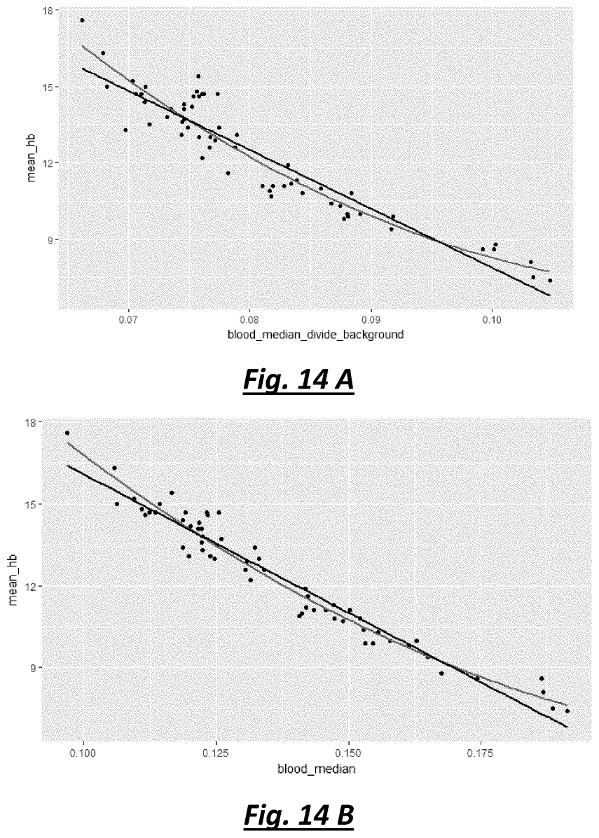

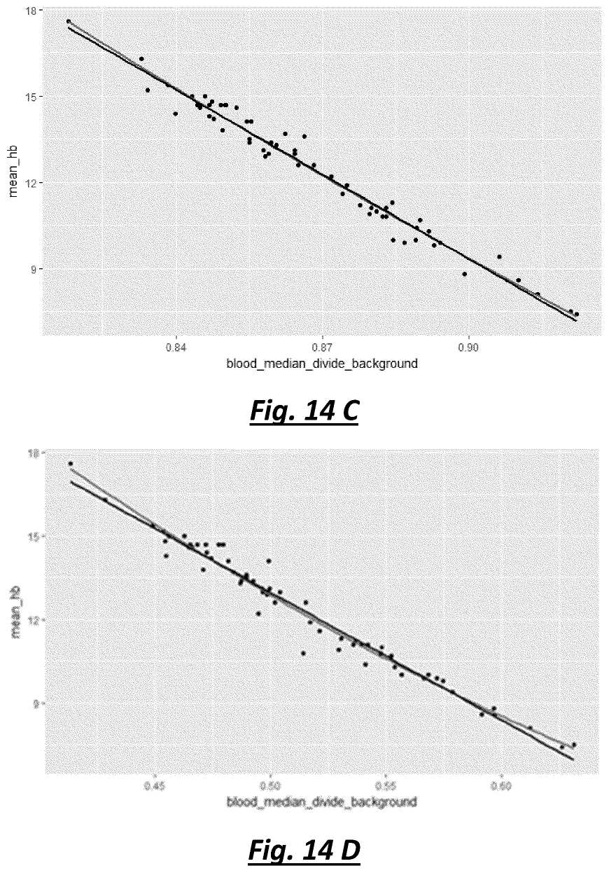

The general construction of the lateral flow assay used in the examples is shown in FIGS. 12 and 13.

Ferritin standards (60, 120, 180, 240, 300, 360, 420, 480, 540 and 600 ng/ml, and 1, 10, 100 and 1000 ng/ml) were prepared by diluting human liver ferritin (Code: P103-7, BBI Solutions) in a buffer consisting of 10 mM Tris-HCl, 140 mM NaCl, 1 ml/L ProClin 950, 1% BSA, pH 7.4.

Anti-ferritin antibodies (IgG) were obtained from BBI Solutions (Ferritin pAb Code: BP230-3) and Europium conjugated with anti-ferritin antibodies were prepared using the Europium conjugation kit from Expedeon Ltd., UK, according to the manufacturer's protocol. A solution of ˜2.3×10E10 Europium particles/ml (0.01%) was used, and stored in a buffer containing 2 mM borate, 10% trehalose, 1 ml/L ProClin 950, at pH 9.5. A conjugate pad (2) was prepared by soaking a glass fiber pad (GFCP203000, Millipore/Merck KGaA, Germany) in this solution and subsequently dried overnight in an oven at 37° C.

In a second example, the conjugate pad was prepared with anti-human-ferritin-conjugated colloidal gold. Gold nanoparticles (InnovaCoat® GOLD—20 OD 80 nm gold conjugation kit, Expedeon Ltd., UK), was conjugated to anti-ferritin antibodies (BBI Solutions, Code no. BP230-3) according to the manufacturer's protocol. The conjugated nanoparticles were diluted to 1.1×10E10 particles/ml (OD=1) using a buffer consisting of 2 mM borate, 10% trehalose, 1 ml/L ProClin 950, at pH 9.5. A conjugate pad (2) was prepared by soaking a glass fiber pad (GFCP203000, Millipore/Merck KGaA, Germany) in this solution and subsequently dried overnight in an oven at 37° C.

In a third example, the conjugate pad was prepared with anti-human-ferritin-conjugated colored latex beads. Black latex beads (Latex conjugation kit—400 nm Black, Expedeon Ltd., UK), was conjugated to anti-ferritin antibodies (BBI Solutions, Code no. BP230-3) according to the manufacturer's protocol. The conjugated latex beads were diluted to ˜2.8×10E9 particles/ml (0,01%) using a buffer consisting of 2 mM borate, 10% trehalose, 1 ml/L ProClin 950, at pH 9.5. A conjugate pad (2) was prepared by soaking a glass fiber pad (GFCP203000, Millipore/Merck KGaA, Germany) in this solution and subsequently dried overnight in an oven at 37° C.

As the sample pad (1), two different glass fiber filters, the LF1 and MF1 (both from GE Healthcare), a cellulose fiber filter (CFSP001700, Millipore/Merck KGaA, Germany) and a combination of a chromatography paper (CHR17 or 31ET from GE Healthcare) and a glass fiber filter (MF1 from GE healthcare) were investigated. The cellulose fiber filter was mainly used for the initial studies using ferritin in buffer. In the last example of sample pad, i.e. a combination of chromatography paper (CHR17 or 31ET) and glass fiber filter (MF1), both components were mounted in the area for sample application on the backing card. The width of the chromatography paper was 12 mm and it was mounted with 2 mm overlap on a glass fiber with a width of 7 mm. This set-up was used for quantification of Hb and ferritin on a single test strip and using the same blood sample.

A lateral flow test strip was constructed by attaching a sample pad (1), a conjugate pad (2), and an absorbent pad (6) onto a membrane backing card (10) with a pre-attached membrane (3) chosen from two different nitrocellulose membranes (Hi-Flow Plus 90, HF090MC100, and Hi-Flow Plus 180, HF180MC100, both from Millipore/Merck KGaA, Germany) where 90 and 180 indicate the wicking rates, i.e. it takes the liquid 90 or 180 seconds to travel 4 cm across the membrane. The different components were assembled with approx. 2 mm overlap to ensure good wicking.

A test line (4) and control line (5) were printed on the membrane (3) using an EASY PRINTER™ from MDI Membrane Technologies Ltd., India, using a solution of 1 mg/ml anti-ferritin (IgG) for the test line (4) and 1 mg/ml anti-IgG (goat anti-rabbit IgG, sigma Aldrich code no. SAB3700883—2 mg) for the control line (5). The membrane and backing card were dried overnight in an oven at 37° C., sprayed with a blocking solution (SuperBlock™ T20 (TBS) Blocking Buffer, code no. 37536, Thermo Fisher Scientific), and dried at 37° C. for an additional 2 hours. This produced an intermediate product a shown in FIG. 12.

Thin strips, approx. 4 mm wide and approx. 60 mm long, were cut using a paper cutter. These strips were placed in a plastic housing as shown in FIG. 13. The plastic housing (200) had a sample port (201) exposing part of the sample pad (1) and a window (202) exposing the test line (4) and control line (5).

### 6.1 Liver Ferritin Standards

50 μl samples were added to the sample pad, followed by an addition of 50 μl of a “chase buffer” of 100 mM Tris-HCl, 50 mM NaCl, 1.5% Tween, 1% BSA, pH 7.4 using an automatic pipette.

For detection using anti-human-ferritin-conjugated Europium beads, the membrane was illuminated with a UV LED producing light at approximately 365 nm, and the emission was measured at approximately 610 nm using a CCD sensor equipped with a dichroic filter. A reading was taken after 5 minutes. The results indicate a good sensitivity at the relevant concentration interval, 100 ng/ml.

For detection using anti-human-ferritin-conjugated gold nanoparticles, the membrane was illuminated with a LED producing light at approximately 525 nm, and reflected light was captured with a CCD sensor. A reading was taken after 5 minutes. Preliminary results indicate a good sensitivity at the relevant concentration interval, 100 ng/ml.

For detection using anti-human-ferritin-conjugated colored latex beads, the membrane was illuminated with a LED producing light at approximately 525 nm, and reflected light was captured with a CCD sensor. A reading was taken after 5 minutes. Preliminary results indicate a good sensitivity at the relevant concentration interval, 100 ng/ml.

The test line intensity showed an almost linear correlation to the concentrations. Additionally, it can be noted that the control line stayed practically constant, indicating that aggregation of Eu-particles is not likely to be pronounced. Experiments with time-resolved measurements indicate that also this approach would be feasible.

### 6.2 Blood Samples—Ferritin Detection Only

Whole blood samples were obtained from healthy volunteers and tested using the lateral flow assay disclosed herein. The blood sample (25 μl) was transferred to the sample pad (MF1, GE healthcare) using a capillary. 75 μl chase buffer (70 mM Tris-HCl, 80 mM NaCl, 1% tween 20, 1% BSA, 0,01% proClin 950, pH 7,4) was applied to the sample pad using a pipette. After 5 minutes the intensity of test line and control line was evaluated using a CCD camera.

For detection using anti-human-ferritin-conjugated Europium beads, the membrane was illuminated with a UV LED producing light at approximately 365 nm, and the emission was measured at approximately 610 nm using a CCD sensor equipped with a dichroic filter. The plasma ferritin concentration in this sample was evaluated using Randox ferritin immunoassay (https://www.randox.com/ferritin/) on Architect c4000, a clinical chemistry analyzer (Abbot Core Laboratory, Abbot Park, Ill., USA).

For detection using anti-human-ferritin-conjugated gold nanoparticles, the membrane was illuminated with a LED producing light at approximately 525 nm, and the reflected light was captured with a CCD sensor. The plasma ferritin concentration in this sample was evaluated to using Randox ferritin immunoassay (https://www.randox.com/ferritin/) on Architect c4000.

For detection using anti-human-ferritin-conjugated colored latex beads, the membrane was illuminated with a LED producing light at approximately 525 nm, and the reflection was measured using a CCD sensor. The plasma ferritin concentration in this sample was evaluated using Randox ferritin immunoassay (https://www.randox.com/ferritin/) on Architect c4000.

6.3 Blood Samples with Detection of Both Hb and Ferritin on the Same Test Strip

Whole blood samples were obtained from healthy volunteers and tested using the lateral flow assay disclosed herein. The blood sample (25 μl) was transferred to the chromatography paper in combined sample pad (i.e. chromatography paper (CHR17) combined with sample pad (MF1)) using a capillary.

Immediately after blood application, the Hb-level was read on the chromatography paper. The paper was illuminated with warm LED spots (˜2800-3000 K) and the reflection was measured using a CMOS sensor (according to example 1 section 1.1)

After Hb-reading, 75 μl chase buffer (70 mM Tris-HCl, 80 mM NaCl, 1% tween 20, 1% BSA, 0,01% proClin 950, pH 7,4) was applied to the chromatography paper using a pipette. After 5 minutes, the intensity of the ferritin test line and control line was evaluated using a CCD camera. Hb of this sample was determined using average of manual Randox Drabkins and HemoCue 201.

For detection using anti-human-ferritin-conjugated Europium beads, the membrane was illuminated with a UV LED producing light at approximately 365 nm, and the emission was measured at approximately 610 nm using a CCD sensor equipped with a dichroic filter. The plasma ferritin concentration in this sample was evaluated using Randox ferritin immunoassay (https://www.randox.com/ferritin/) on Architect c4000.

For detection using anti-human-ferritin-conjugated gold nanoparticles, the membrane was illuminated with warm LED spots (˜2800-3000 K) and the reflection was measured using a CMOS sensor. The plasma ferritin concentration in this sample was evaluated using Randox ferritin immunoassay (https://www.randox.com/ferritin/) on Architect c4000.

For detection using anti-human-ferritin-conjugated colored latex beads, the membrane was illuminated with a LED producing light at approximately 525 nm, and the reflection was measured using a CCD sensor. The plasma ferritin concentration in this sample was evaluated using Randox ferritin immunoassay (https://www.randox.com/ferritin/) on Architect c4000.

### Discussion

The prototype lateral flow assay confirms the feasibility of the method, and taken together with the results from optical measurements of hemoglobin on filter paper, a combined lateral flow assay for these two analytes appears feasible. As these two analytes are present in highly different concentrations, separated by a magnitude of 10E6, the simultaneous or substantially simultaneous measurement of these two analytes on the same assay device is nothing less than surprising.

### Example 7. Influence of Hct on Ferritin Measurements

A lateral flow assay comprising a glass fiber-based sample pad, and in fluid connection therewith, a conjugate pad with anti-ferritin antibodies, and downstream on a filter medium, immobilized anti-ferritin antibodies or fragments is assembled and tested. Upon application of a whole blood sample on the sample pad, the reflectance and the area of the blood spot is measured within 1-10 seconds from application of the sample. Based on this reading, the hematocrit volume fraction is calculated.

The plasma ferritin concentration is read after an incubation period of about 5 minutes, and the ferritin concentration presented with consideration of the previously calculated Hct for the sample. Assuming a normal distribution of the Hct for men and women (within the intervals indicated by Henny H. Billett, 1990, ibid) the use of the predicted (calculated) Hct instead of an average for each gender, or an average for all patients, resulted in an improved accuracy for a majority of patients.

### Example 8. Plasma Calprotectin

Calprotectin in plasma and blood is a useful biomarker of inflammation and infection, and a POC test for determining calprotectin in a whole blood sample would be a significant improvement. Here it however becomes extremely important to take the true Hct into account, as variations in Hct will influence the amount of plasma available for the assay. In a theoretical example, the inventors postulate that a calprotectin value in plasma of 1.5 mg/l can be significantly under—as well as overestimated in cases of high or low Hct values, values still within the normal ranges for adult men and women.

As shown in the table below, a true value of 1.5 mg/ml calprotectin in plasma will be displayed as 1.30 mg/l for male patients having a high Hct while still in the normal Hct range. Conversely, the value will be significantly overestimated for patients having low Hct, 1.70 mg/l for male patients, and 1.66 mg/l for female patients having a low Hct. Such errors may lead to an incorrect diagnosis.

For patients with Hct values outside the normal range, the error will be even more significant.

TABLE 3
Plasma calprotectin

| | Males | Females |
|---|---|---|
| Hct range (mean) | 40-54% (47%) | 36-48% (42%) |

Example given: 1.50 mg/l

| | | |
|---|---|---|
| HCT low | 1.70 mg/l | 1.66 mg/l |
| HCT mean | 1.50 mg/l | 1.50 mg/l |
| HCT high | 1.30 mg/l | 1.34 mg/l |

The above was later confirmed in practical experiments, using a lateral flow assay platform, equipped with a glass fiber filter for separating plasma, and for allowing the instantaneous measurement of Hct. The results are presented in Example 13.

### Example 9. Cystatin C

Cystatin C is a small protein with a basic isoelectric point that has emerged as an alternative marker for kidney function. The justification for the use of cystatin C as a marker for renal function follows the same basic logic as that for creatinine. Since cystatin C is not secreted and does not return to the blood stream but rather is reabsorbed by tubular epithelial cells and subsequently degraded, it avoids some of the non-renal effectors such as muscle mass, age or gender that complicate the use of other endogenous markers.

It has been shown that increased levels of cystatin C are associated with a higher risk of death from all causes, and the highest quintile of cystatin C (≥1.29 mg/l) is associated with a significantly elevated risk of death from cardiovascular causes, myocardial infarction, and stroke after multivariate adjustment. It was concluded that cystatin C is a stronger predictor of the risk of death and cardiovascular events in elderly persons than is creatinine (Shiplak M. G. et al., Cystatin C and the Risk of Death and Cardiovascular Events among Elderly Persons, May 19, 2005, N Engl J Med 2005; 352:2049-2060, DOI: 10.1056/NEJMoa043161

The inventors postulate that a Cystatin C value in plasma can be significantly under-as well as overestimated in cases of high or low Hct values, values still within the normal ranges for adult men and women. As shown in Table 4 below, a true value of 1.03 mg/ml in plasma will be displayed as 0.89 mg/I for male patients, and 0.92 mg/l for female patients having a high Hct while still in the normal Hct range. Conversely, the value will be significantly overestimated for patients having low Hct. Such errors may lead to an incorrect diagnosis. Unidentified outliers, i.e. patients with a very low HCT or very high HCT will be subject to very inaccurate measurement of plasma cystatin C values, and an inaccurate estimation of glomerular filtration rates.

TABLE 4
Cystatin C

| | Males | Females |
|---|---|---|
| Hct range (mean) | 40-54% (47%) | 36 - 48% (42%) |
| Cystatin C plasma value | 0.57 mg/l | 0.57 mg/l |
| HCT low | 0.65 mg/l | 0.63 mg/l |
| HCT mean | 0.57 mg/l | 0.57 mg/l |
| HCT high | 0.49 mg/l | 0.51 mg/l |
| Cystatin C plasma value | 1.03 mg/l | 1.03 mg/l |
| HCT low | 1.17 mg/l | 1.14 mg/l |
| HCT mean | 1.03 mg/l | 1.03 mg/l |
| HCT high | 0.89 mg/l | 0.92 mg/l |
| Cystatin C plasma value | 1.06 mg/l | 1.06 mg/l |
| HCT low | 1.20 mg/l | 1.17 mg/l |
| HCT mean | 1.06 mg/l | 1.06 mg/l |
| HCT high | 0.92 mg/l | 0.95 mg/l |

### Example 10. Ferritin

It is generally known that the ferritin concentration in plasma reflects the size of the iron reserve in the body. Ferritin has been studied in large-scale surveys of the iron status of populations. It has also been found useful in the assessment of clinical disorders of iron metabolism. A low plasma ferritin level has a high predictive value for the diagnosis of uncomplicated iron deficiency anemia. The normal range for blood ferritin is 20 to 500 nanograms per millilitre (for adult men) and 20 to 200 nanograms per millilitre (for adult women). Recently, the ferritin-to-hemoglobin ratio has been suggested as a useful tool to predict survival in patients with advanced non-small-cell lung cancer (NSCLC). The ferritin-to-hemoglobin ratio, a potential parameter of tumor progression, was a significant prognostic factor for overall survival, with a direct correlation to survival time in patients with advanced NSCLC (Sookyung Lee et al., Prognostic Value of Ferritin-to-Hemoglobin Ratio in Patients with Advanced Non-Small-Cell Lung Cancer, J Cancer 2019; 10(7):1717-1725. doi:10.7150/jca.26853).

The inventors postulate that the measurement of ferritin in plasma is highly dependent on the hematocrit, and that the concentration of ferritin can be significantly under-as well as overestimated in cases of high or low Hct values, values still within the normal ranges for adult men and women. This becomes more important as new diagnostic applications of the ferritin concentration are presented. The influence of variations in Hct on ferritin measurements is illustrated in the table below.

TABLE 5
Plasma (liver) ferritin

| | Males | Females |
|---|---|---|
| HCT | 40-54% | 36-48% |
| Ferritin range (μg/l) | 20-300 | 15-200 |
| Low HCT | 23-339.62 | 16.55-220.69 |
| High HCT | 17.36-260.38 | 13.45-179.31 |

Example given: 25 μg/l

| | | |
|---|---|---|
| Low HCT | 28.30 μg/l | 27.59 μg/l |
| High HCT | 21.70 μg/l | 22.41 μg/l |

The effect is even more pronounced for low values, for example ferritin values encountered in different forms of cancer. The inventors postulate that the plasma ferritin value will be overestimated in patients having a low Hct, and underestimated in patients having high Hct, which can result in low ferritin values being overlooked.

### Example 11. PCT—Plasma Procalcitonin

In another theoretical example, the inventors postulate that a plasma procalcitonin value of 0.15 μg/l can be significantly under-as well as overestimated in cases of high or low Hct values, values still within the normal ranges for adult men and women. As shown in the table below, a true value of 0.15 μg/l in plasma will be displayed as 0.13 μg/l for patients having a high Hct while still in the normal Hct range. Conversely, the value will be significantly overestimated (0.17 μg/l) for patients having low Hct. Such errors may lead to an incorrect diagnosis.

For patients with Hct values outside the normal range, the error will be even more significant.

TABLE 6
Plasma procalcitonin

| | Males | Females |
|---|---|---|
| HCT | 40-54% | 36-48% |

Example given: Plasma value 0.15 μg/l

| | | |
|---|---|---|
| Low HCT | 0.17 μg/l | 0.17 μg/l |
| High HCT | 0.13 μg/l | 0.13 μg/l |

General considerations (information from Mayo Clinic Laboratories): In children older than 72 hours and in adults, levels below 0.15 ng/mL make a diagnosis of significant bacterial infection unlikely. Procalcitonin (ProCT) between 0.15 and 2.0 ng/mL does not exclude an infection, because localized infections (without systemic signs) may be associated with such low levels. Levels above 2.0 ng/mL are highly suggestive of systemic bacterial infection/sepsis or severe localized bacterial infection, such as severe pneumonia, meningitis, or peritonitis. They can also occur after severe non-infectious inflammatory stimuli such as major burns, severe trauma, acute multiorgan failure, or major abdominal or cardiothoracic surgery. In cases of non-infectious elevations, ProCT levels should begin to fall after 24 to 48 hours.

Autoimmune diseases, chronic inflammatory processes, viral infections, and mild localized bacterial infections rarely lead to elevations of ProCT of more than 0.5 ng/mL.

### Example 12. C-Reactive Protein—CRP

In this theoretical example, the inventors postulate that a CRP value in plasma of 5 mg/l can be significantly under-as well as overestimated in cases of high or low Hct values, values still within the normal ranges for adult men and women. As shown in the table below, a true value of 5 mg/ml in plasma will be displayed as 4.4 mg/l for male patients having a high Hct while still in the normal Hct range. Conversely, the value will be significantly overestimated for patients having low Hct. Such errors may lead to an incorrect diagnosis.

For patients with Hct values outside the normal range, the error will be even more significant. This may have significant consequences when conducting a high-sensitive CRP test (hs-CRP). An hs-CRP test is performed to evaluate a patient's risk of heart disease. The current risk levels used are:

An hs-CRP level of less than 2.0 milligram per liter (mg/L) indicates a lower risk, while an hs-CRP level greater than 2.0 mg/L indicates an increased risk.

The below table indicates that there is a risk that the hs-CRP values will be underestimated in patients having a high Hct. This in turn could lead to that patients running a risk for contracting heart diseases would not be identified. For patients with even higher Hct, so called outliers, the error will be even greater.

TABLE 7
C-reactive protein

| | Males | Females |
|---|---|---|
| HCT | 40-54% | 36-48% |

Example given: 5 mg/l

| | | |
|---|---|---|
| Low HCT | 5.66 mg/l | 5.52 mg/l |
| High HCT | 4.34 mg/l | 4.48 mg/l |

### Example 13. Plasma Calprotectin Measured in Whole Blood Samples

A commercial lateral flow assay for quantifying serum calprotectin (Quantum Blue® sCAL/MRP8/14, Bühlmann Laboratories AG, Switzerland) was modified as follows: The test cassette was carefully opened and the lateral flow strip removed. The sample pad was removed from the conjugate pad and replaced with a VF2 blood filter (a bound glass fibre filter from GE Healthcare) of the same width and length. The cassette was reassembled. The general construction of the cassette was basically as shown in FIG. 13, wherein the sample pad (1) was replaced.

A total of 40 whole blood samples of varying Hct and calprotectin levels were obtained from healthy volunteers. The Hct of each sample was assigned by centrifugation (Haematokrit 200 centrifuge, Andreas Hettich GmbH & Co.KG, Tuttlingen, Germany). A sample of 10 μl whole blood was pipetted onto the VF2 filter and imaged through the regular sample addition port or well of the sCAL test cassette. A Pixelink PL-D795MU-5MP monochrome camera (5 MP, 2/3 sensor) and a VZM 100i video lens (Edmund Optics) was used for imaging the blood spots. An 800 nm band pass filter (Thorlabs; FB800-10) was used. The images were taken as 16 bit TIF (2048×2448 pixels) without gamma correction. Exposure time 1500 ms. The sample was illuminated with two 50 W LED spots, having a colour temperature of about 2700 K. 5 blank images were taken, and averaged (pixel-by-pixel) to define the illumination profile, prior to imaging the blood spots. The sample and camera were enclosed with a blackout material (Thorlabs) to prevent changing external light conditions from influencing the images.

As a first step in the post-processing, all images of the blood spots were divided by the illumination profile (pixel-by-pixel). Then the pixels constituting the blood spot were identified using segmentation. The number of pixels constituting the blood spot was used as the measure of the “blood spot area”. A circle with a radius of 250 pixel placed at the spot's “center of mass” defined the area in which the median pixel value was calculated.

Both blood spot area and median pixel value (at the blood spot center) were used to predict the Hct level of the blood sample.

When the imaging of the blood spot had been performed, 90 μl chase buffer (supplied in the sCAL kit, ibid) was added and the calprotectin concentration determined after 12 minutes, using a commercial lateral flow test reader (Quantum Blue®, Bühlmann Laboratories AG, Switzerland), according to the manufacturer's instructions.

It was also investigated how the modification of the commercial lateral flow device influenced its performance. First, the inventors added the same amount of calprotectin to measure 4.7 μg/ml in an unmodified device: [60 μl (high control: 4.7 μg/ml)+36 μl chase buffer] was added to the modified test. This was repeated three times and the following results obtained: 2.26 μg/ml, 1.65 μg/ml and 2.11 μg/ml.

Then, the same amount of calprotectin was added to measure 2.35 μg/ml in the unmodified test: [30 μl (high control: 4.7 μg/ml)+66 μl chase buffer] was added to the modified test. This was also repeated three times and the following results obtained: 1.07 μg/ml, 1.39 μg/ml and 1.05 μg/ml.

These six measurements indicate that the modified test give (on average) a smaller concentration by a factor 2.18. Hence, all values measured in the modified test were multiplied with 2.18 before being compared to Gentian's turbidimetric results.

It was seen that the modified lateral flow test handled whole blood samples well. The modified test measured calprotectin concentrations (in whole blood) in the same ball park as Gentian's turbidimetric reference method in plasma (GCAL®, Gentian AS, Norway). In this first set of experiments, the deviations were fairly high compared to the unmodified lateral flow test for calprotectin in serum (sCAL, modified as described above).

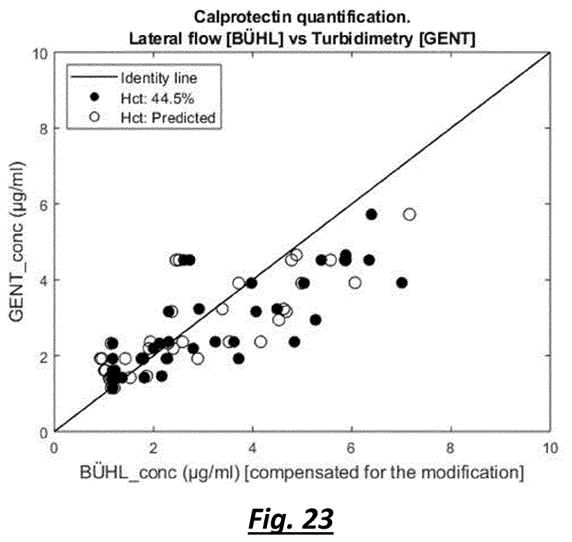

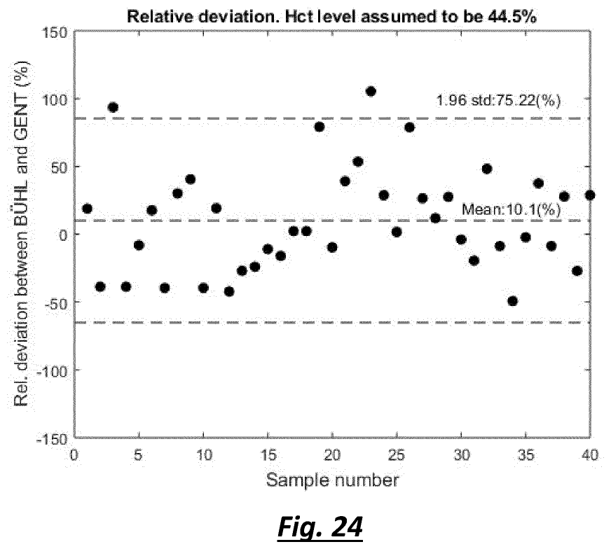

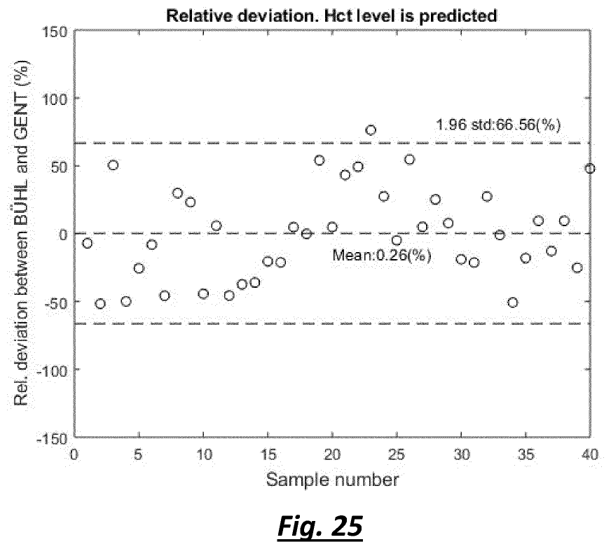

However, the average deviations were close to zero, which is promising. Assuming a constant Hct level for all samples of 44.5% gives a variation in measured calprotectin concentration of: 1.96std=2.21 μg/ml or 75.22%. The average deviation was 0.30 μg/ml or 10.10%. When determining the Hct based on the optical imaging of the blood spot, and applying the Hct level to the calprotectin results, the deviation is significantly reduced. In this experiment, a variation in measured calprotectin concentration of 1.96std=2.02 μg/ml or 66.56% was recorded. The average deviation was 0.08 μg/ml or 0.26%. The results are shown in FIGS. 23-25.

Hct prediction and compensation is thus shown to have a positive effect on the variation in the measured calprotectin concentration. It is likely that a greater effect is achieved using a professionally assembled strip test.

### Example 14. Combination of Area and Intensity Measurement for the Determination of Hematocrit

A total of 10 samples with different Hct levels were prepared as follows. Three 4 ml Li-Heparin tubes were filled with venous blood from a healthy donor. The tubes were centrifuged at 1000 g for 10 min at 4 C to precipitate the RBC. Plasma and RBCs were separated and mixed in various proportions to prepare 10 samples with Hct levels ranging from 20.85 to 58.50%, covering the physiological range of 25-60%. Each sample had a total volume of 600 μl. The true Hct of each sample was assigned based on the average of two measurements, using the Haematocrit 200 device (Hettich).

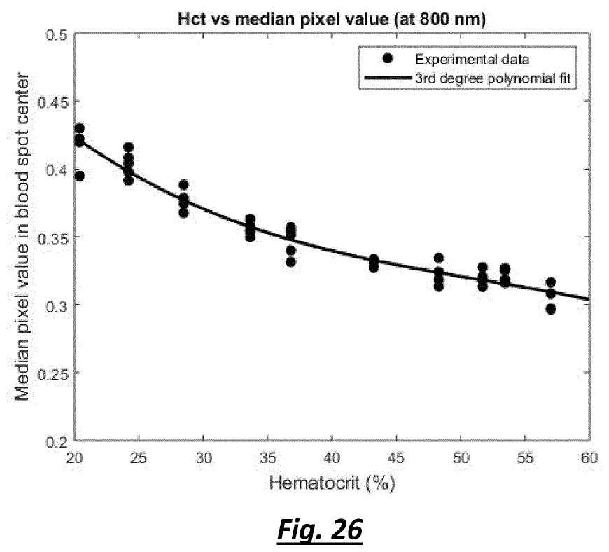

For each Hct level, five blood spots (10 μl) on VF2 (5×20 mm) glass fiber filter paper were produced, and imagined using an 800 nm optical filter, exposure time 1500 ms. The blood spots were identified by comparison with the background (image without blood), a region-of-interest (ROI) circle was placed at the center of mass of the blood spot, and the median pixed value within the circle as calculated. The area of the entire blood spot was also calculated. The median pixel value (at 800 nm) at the ROI after background correction correlated well with the assigned Hct value. A prediction model based on a 3rd degree polynomial exhibited a relative deviation to the assigned values of 14.24% (1.96 standard deviations. See FIG. 26.

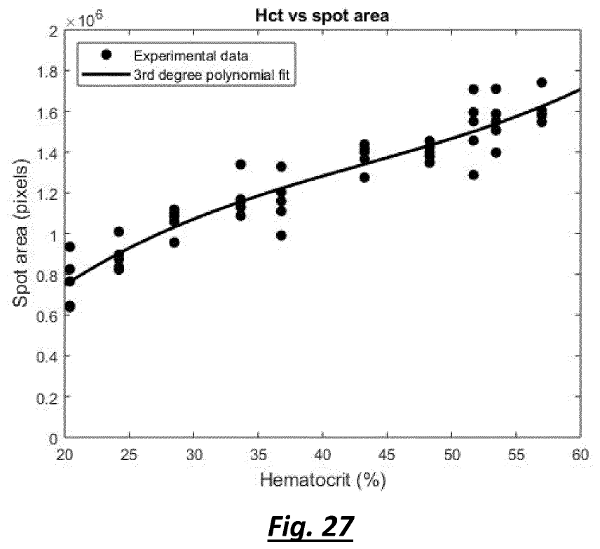

The blood spot area was also shown to correlate with the assigned Hct value. A prediction model based on a 3rd degree polynomial exhibited a relative deviation to the assigned values of 20.38% (1.96 standard deviations). See FIG. 27.

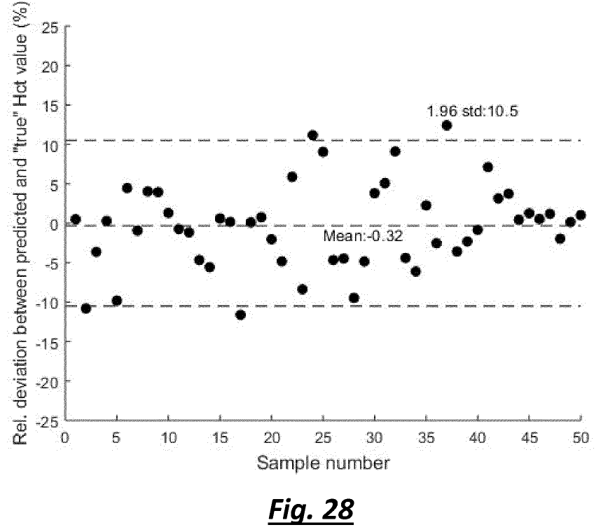

However, a combination of the two models, i.e. averaging the predictions from the two models exhibited a relative to the assigned values of ˜10.50% (1.96 standard deviations). See FIG. 28 which shows the improved precision (1.96 std=10.50%) achieved by formulating a Hct prediction model that averages the predictions from two separate Hct prediction models based on 1) median pixel intensity and 2) blood spot area. These two separate Hct prediction models alone had precisions of 1.96 std=14.24% and 20.38%, respectively.

Without wishing to be bound by theory, the present inventors believe that the combination of these two models compensates for the bias caused by changes in color, viscosity or rheology of whole blood at low and high Hct respectively, and that the combination of the two offers a highly accurate approach to optical determination of Hct.

Without further elaboration, it is believed that a person skilled in the art can, using the present description, including the examples, utilize the present invention to its fullest extent. Also, although the invention has been described herein with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-12. (canceled)

13. A lateral flow assay method for determining the concentration of an analyte in a sample of whole blood, comprising the following steps:
a) applying an untreated sample of whole blood to a first surface in said lateral flow assay to form a blood spot thereon;
b) taking an image of said blood spot within 1-300 seconds after the applying step;
c) analysing said image to determine a first value indicative of the hematocrit level of said sample;
d) determining a second value indicative of the amount of the analyte in the sample; and determining the concentration of the analyte in the sample based on the hematocrit level determined in step c) and the amount of analyte determined in step d).

14. The lateral flow assay method of claim 13, wherein the analyte is chosen from ferritin, transferrin, plasma calprotectin, C-reactive protein (CRP), cystatin C, plasma procalcitonin (PCT) and anti-CCP antibodies.

15. The lateral flow assay method according to claim 13, wherein said image is taken within 1-180 seconds, preferably 1-120 seconds, more preferably within 1-30 seconds, and most preferably within 1-10 seconds after the applying step (a).

16. The lateral flow assay method according to claim 13, wherein the first value determined in step (c) is a reflectance of said blood spot or an area of said blood spot.

17. The lateral flow assay method according to claim 16, wherein both the reflectance of said blood spot and the area of said blood spot are determined and correlated to a preliminary hematocrit level, and the average of the two is used as the value of the hematocrit level.

18. The lateral flow assay method according to claim 16, wherein the reflectance value is determined at at least one wavelength in a range from 390 nm to 1000 nm, preferably in the interval of 650 nm to 1000 nm, for example at at least one wavelength chosen from 660 nm, 780 nm, 800 nm, and 940 nm.

19. The lateral flow assay method according to claim 16, wherein the reflectance is determined at a wavelength of 800 nm.

20. The lateral flow assay method according to claim 16, wherein the reflectance is determined at wavelengths of 660 nm and 940 nm.

21. The lateral flow assay method according to claim 16, wherein the reflectance is measured as the median intensity of the pixels included in said image taken in step (b) using an 800 nm optical filter.

22. The lateral flow assay method according to claim 13, further comprising a calibration step by means of which a reference hematocrit level of a reference sample is determined by centrifugation.

23. The method according to claim 13, wherein the hematocrit level is determined by first optically determining a concentration of hemoglobin in said sample and then converting said hemoglobin concentration into the hematocrit level.

24. The method according to claim 23, wherein the hemoglobin concentration is converted into the hematocrit level by multiplying the hemoglobin concentration in g/dl by a factor of 3, thus yielding the hematocrit level in %.

25-35. (canceled)

36. A system for determining the concentration of an analyte chosen from plasma calprotectin, cystatin C, ferritin, plasma procalcitonin (PCT), C-reactive protein (CRP), and anti-CCP antibodies in a whole blood sample, wherein said system comprises a lateral flow assay device having a substrate configured to form a blood spot thereon upon application of a sample of whole blood onto the substrate, a light source, a detector arranged to detect light reflected from the blood spot and to determine a reflectance and/or a size of the blood spot, and a processor configured to correlate the reflectance and/or the size of the blood spot to the hematocrit level of said sample, and configured to take the measured Hct into account when determining the concentration of said analyte.

37. A system for determining the concentration of an analyte chosen from plasma calprotectin, cystatin C, ferritin, plasma procalcitonin (PCT), C-reactive protein (CRP), and anti-CCP antibodies in a whole blood sample, wherein said system comprises a lateral flow assay device having a substrate configured to form a blood spot thereon upon application of a sample of whole blood onto the substrate, a light source, a detector arranged to detect light reflected from the blood spot and to determine the reflectance and/or a size of the blood spot, a processor configured to correlate the reflectance and/or the size of the blood spot to a hemoglobin concentration of said sample based on values of the reflectance and/or the size obtained for known hemoglobin concentrations, and to calculate the hematocrit level, and configured to take the calculated Het into account when determining the concentration of said analyte.

38. A device for receiving a lateral flow assay device for determining the concentration of an analyte chosen from plasma calprotectin, cystatin C, ferritin, plasma procalcitonin (PCT), C-reactive protein (CRP), and anti-CCP antibodies in a whole blood sample, comprising at least a light source, a detector arranged to detect reflected light and/or to determine the size of a blood spot formed on a substrate of said lateral flow device, and a processor configured to correlate the reflectance and/or size of the blood spot to a hematocrit level and to use this hematocrit level when calculating the concentration of said analyte in a sample of whole blood.
